(19) United States
(12) Patent Application Publication
(10) Pub. No.: US 2024/0190878 A1
(43) Pub. Date: Jun. 13, 2024

(54) ANTIVIRAL PRODRUGS OF ETV AND FORMULATIONS THEREOF

(71) Applicant: THE SCRIPPS RESEARCH INSTITUTE, La Jolla, CA (US)

(72) Inventors: Arnab Chatterjee, San Diego, CA (US); Anil Kumar Gupta, San Diego, CA (US); Anders Mikal Eliasen, Los Angeles, CA (US)

(21) Appl. No.: 18/550,500

(22) PCT Filed: Mar. 14, 2022
(86) PCT No.: PCT/US22/71137

§ 371 Date: Sep. 14, 2023

(51) Int. Cl. C07D 473/18; A61K 9/08; A61K 31/522; A61K 45/06; A61K 47/34; A61K 47/38; A61K 47/44

(52) U.S. Cl. CPC C07D 473/18; A61K 9/08; A61K 31/522; A61K 45/06; A61K 47/34; A61K 47/38; A61K 47/44

(57) ABSTRACT

Provided herein are compounds, compositions, and their methods of use for treatment and/or prevention of infections of HBV in a subject by administering a compound of structural formula (I) or a pharmaceutically acceptable salt thereof: wherein R¹ and R² are defined herein.

This application claims the benefit of priority to U.S. Provisional Patent Application No. 63/161,719 filed on Mar. 16, 2021, which application is incorporated in its entirety as if fully set forth herein.

### BACKGROUND

Chronic hepatitis B virus (HBV) infection is a major public health problem that affects millions of people worldwide every year. Certain nucleoside analogue reverse transcriptase (RT) inhibitors, such as Entecavir (ETV) have high barrier against HBV resistance (Curr Med Res Opin 2005, 27 (11), 1845-1856.)

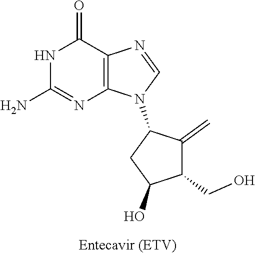 [SMILES: C=C1[C@H](CO)[C@@H](O)C[C@@H]1n1cnc2c(=O)[nH]c(N)nc21]

Reverse transcriptase inhibitors can be effective in the treatment of viral infections caused by viruses where reverse transcriptase function is essential for viral replication and the production of viral proteins, including HBV. In the case of HBV, which is a DNA virus, the DNA viral polymerase also has a reverse transcriptase function, generating viral DNA from a viral RNA intermediate during replication. An antiviral example of a compound that treats HBV is lamivudine.

Because patient compliance challenges reside in the daily dosing of ETV, efforts have been made to facilitate sustained release of ETV. For example, a prodrug of ETV is ETV-palmitate, which demonstrates plasma levels above therapeutic level for up to 1 month (Int. J. Pharm. 2018, 543 (1), 52-59.).

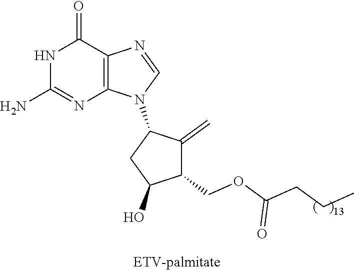

### SUMMARY

To address this challenge and others, the present disclosure provides, in various embodiments, a bioactive prodrug of ETV according to formula (1) or a pharmaceutically acceptable salt thereof:

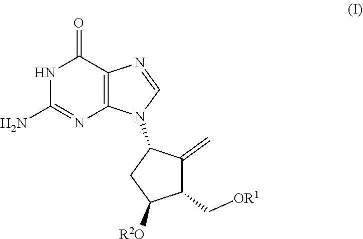

wherein
R¹ is H or is X-Lₘ,
m=1 or 2;
X-Lₘ is —C(═O)L, —C(═O)OL, —C(═O)NH(L), —C(═O)N(L)₂, —CH(R)OC(═O)L, —C(═O)CH(R)—NH(L), —C(═O)CH(R)—N(L)₂, —P(═O)(NHL)₂, —P(═O)(NHL)(NL₂), or —P(═O)(NL₂)₂,
each L is independently selected from (C1-22, linear and branched)alkyl, (C3-22, linear and branched)alkenyl, (C3-7)cycloalkyl, (CHR)ₙ-phenyl wherein n=0 or 1, and —CHR—N(R)2;
or R¹ is —OCH(R)OP(═O)(OH)₂, a phosphate residue or its derivative residue comprising a monophosphate, a diphosphate, a triphosphate, a phosphonate, a phosphate polyester, a phosphate amidate (mono and di), a phosphorothioate, a phosphoroselenoate, or a phosphoroboranoate;
R is H, (C1-22)alkyl, (C3-22)alkenyl, (C3-7)cycloalkyl, or (C6-C12)aryl;
R² is H or is X-Lₘ;
or R² is —OCH(R)OP(═O)(OH)₂, a phosphate residue or its derivative residue comprising a monophosphate, a diphosphate, a triphosphate, a phosphonate, a phosphate polyester, a phosphate amidate (mono and di), a phosphorothioate, a phosphoroselenoate, or a phosphoroboranoate,
and wherein R¹ and R² are not simultaneously H

The present disclosure also provides in additional embodiments a pharmaceutical composition comprising a compound or pharmaceutically acceptable salt thereof as described herein, and a pharmaceutically acceptable carrier.

In additional embodiments, the present disclosure provides a formulation comprising a compound or pharmaceutically acceptable salt thereof as described herein. The compound is suspended in an aqueous solution comprising a water-soluble cellulose-based polymer and a non-ionic surfactant.

Also provided in another embodiment is a method of inhibiting viral reverse transcriptase bioactivity. The method comprises contacting a virus expressing an enzyme with reverse transcriptase bioactivity with an effective amount or concentration of a compound or a pharmaceutically acceptable salt thereof as described herein.

In further embodiments, the present disclosure provides method for treatment or prevention of a viral infection in a patient, such as an infection wherein inhibition of a reverse transcriptase is medically indicated. The method comprises administering to the patient in need of treatment or prevention an effective amount of a compound or a pharmaceutically acceptable salt thereof as described herein.

### BRIEF DESCRIPTION OF THE DRAWINGS

FIG. 1. Differential Scanning Calorimetry (DSC) of ETV.

FIG. 2. X-ray Powder Diffractogram (XPRD) of ETV.

FIG. 3. X-ray Powder Diffractogram (XPRD) of micronized ETV and its formulation.

FIG. 4. Differential Scanning Calorimetry (DSC) of Prodrug 9.

FIG. 5. X-ray Powder Diffractogram (XPRD) of Prodrug 9.

FIG. 6. Differential Scanning Calorimetry (DSC) of Prodrug 11.

FIG. 7. X-ray Powder Diffractogram (XPRD) of Prodrug 11.

FIG. 8. X-ray Powder Diffractogram (XPRD) of formulated Prodrug 11.

FIG. 9. Differential Scanning Calorimetry (DSC) of Prodrug 36.

FIG. 10. X-ray Powder Diffractogram (XPRD) of Prodrug 36.

FIG. 11. X-ray Powder Diffractogram (XPRD) of micronized Prodrug 36 and its formulation.

FIG. 12. Differential Scanning Calorimetry (DSC) of Prodrug 29.

FIG. 13. X-ray Powder Diffractogram (XPRD) of Prodrug 29.

FIG. 14. X-ray Powder Diffractogram (XPRD) of micronized Prodrug 29 and its formulation.

FIG. 15. Differential Scanning Calorimetry (DSC) of ETV-palmitate.

FIG. 16. X-ray Powder Diffractogram (XPRD) of ETV-palmitate.

FIG. 17. X-ray Powder Diffractogram (XPRD) of micronized ETV-palmitate and its formulation.

FIG. 18. Rat PK data for ETV and prodrug 9 following IM administration (Example 39; Table 2).

FIG. 19. Dog PK data for prodrug 11, 29 and ETV following IM administration (Example 40; Table 3).

FIG. 20. Dog PK data for prodrug 11, 36 and ETV-palmitate following IM/SC administration (Example 41; Table 4).

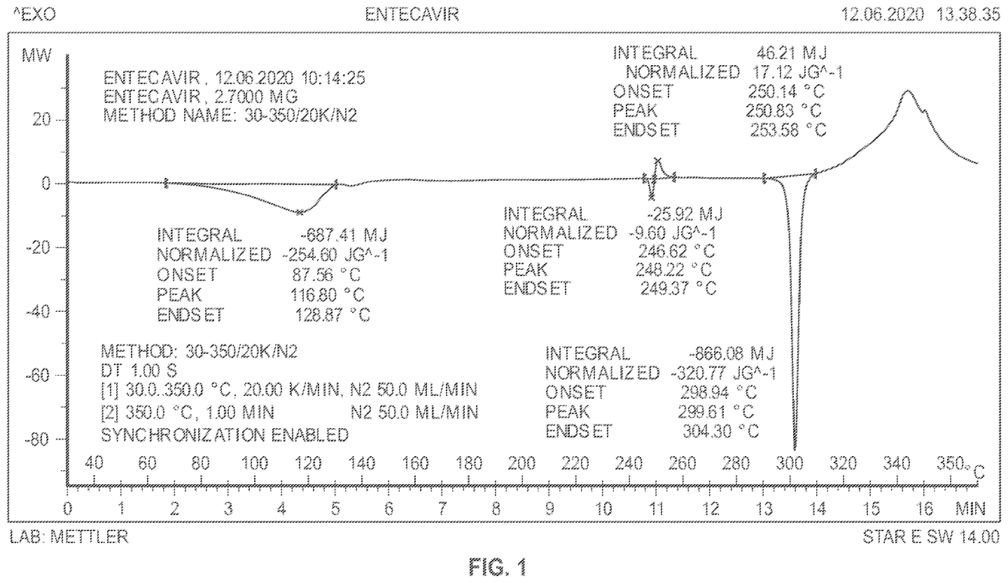

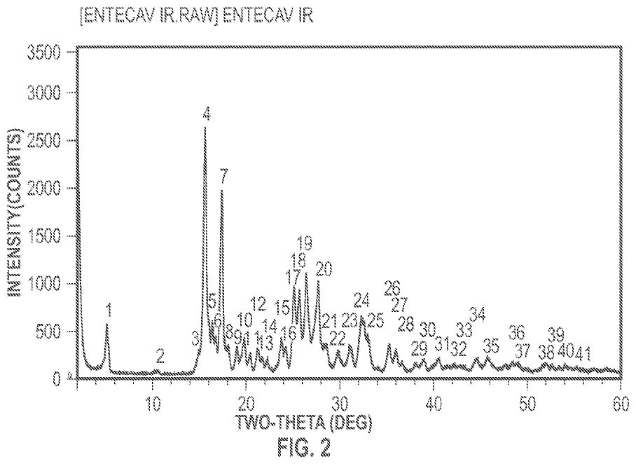

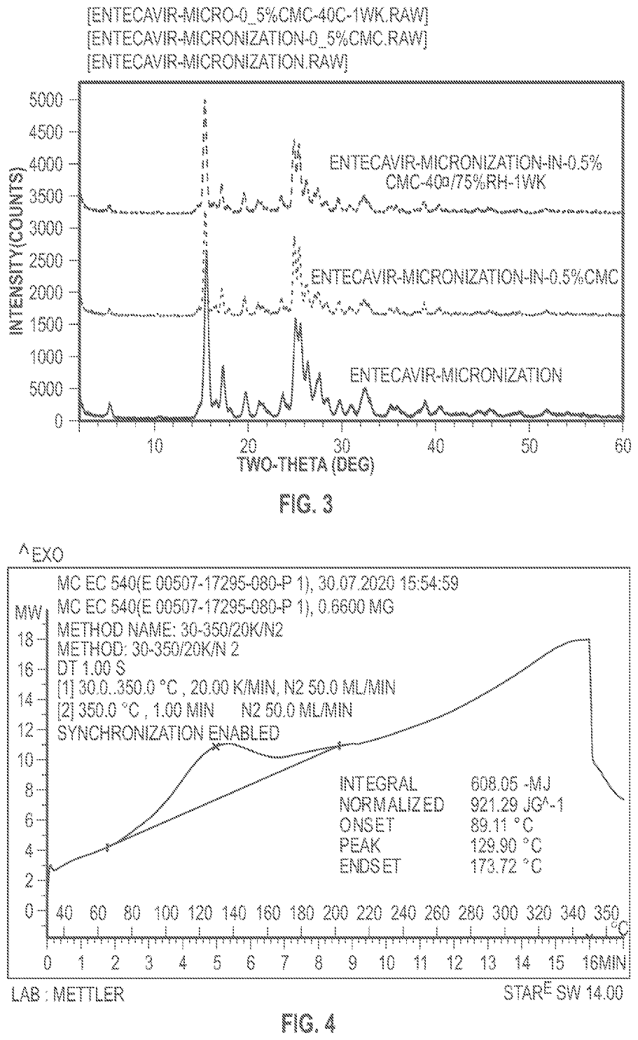

### DETAILED DESCRIPTION

The present disclosure relates in part to compounds that are prodrugs of ETV. Further, formulations of these prodrugs provide therapeutic and prophylactic treatment of patients against viral infections of viruses such as HBV, wherein inhibition of a reverse transcriptase enzyme (RNA-directed DNA polymerase) slows or blocks the viral infection. The route of administration for these treatments can include, but not limited to, oral, parenteral and implants (composition and device). The formulations of the present disclosure provide for slow or controlled or sustained release of ETV from these prodrugs, such as when injected as an aqueous suspension and/or oil solution formulation, thus enabling the inventive ETV prodrugs to serve as long-acting agents.

### Definitions

“Alkyl” refers to straight (linear) or branched chain hydrocarbyl including from I to about 25 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, beptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH3)₂, —C(CH₃)₃, —C(CH₂CH₃)₃, —CH₂ CH(CH₃)₂, —CH₂CH(CH₃)(CH₂CH₃), —CH₂CH(CH)CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂C H(CH₂CH₃)₂, —CH₂CH₂C(CH₃)₃, —CH₂CH₂C(CH₂CH₃)₃, —CH(CH₃)CH₂CH(CH₃)₂, —CH(CH₃) CH(CH₃)CH(CH₃)₂, and the like. Thus, alkyl groups include primary alkyl groups, secondary alkyl groups, and tertiary alkyl groups.

The term “alkenyl” refers to straight or branched chain hydrocarbyl groups including from 2 to about 25 carbon atoms having 1 to 10, 1 to 8, 1 to 6, 1 to 4, 1 to 2, or at least one unsaturation, i.e., carbon to carbon double bond.

The term “cycloalkyl” refers to a saturated monocyclic, bicyclic, tricyclic, or polycyclic, 3- to 14-membered ring system, such as a C₃-C₈-cycloalkyl. The cycloalkyl may be attached via any atom. Representative examples of cycloalkyl include, but are not limited to cyclopropyl, cyclobutyl, cyclopentyl, and cyclohexyl. Polycyclic cycloalkyl includes rings that can be fused, bridged, and/or spiro-fused.

“Aryl” when used alone or as part of another term means a carbocyclic aromatic group whether or not fused having the number of carbon atoms designated or if no number is designated, up to 14 carbon atoms, such as a C₆-C₁₀-aryl or C₆-C₁₄-aryl. Examples of aryl groups include phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). “Aryl” also contemplates an aryl ring that is part of a fused polycyclic system, such as aryl fused to cycloalkyl as defined herein. An exemplary aryl is phenyl.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified to the contrary, the term “compound” is inclusive in that it encompasses a compound or a pharmaceutically acceptable salt, stercoisomer, and/or tautomer thereof. Thus, for instance, a compound of the present disclosure includes a pharmaceutically acceptable salt of a tautomer of the compound.

In this description, a “pharmaceutically acceptable salt” is a pharmaceutically acceptable, organic or inorganic acid or base salt of a compound described herein Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, Jactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this example, the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In certain embodiments, such terms refer to minimizing the spread or worsening of the disease resulting from the administration of one or more prophylactic or therapeutic agents to a patient with such a disease.

The terms “prevent,” “preventing.” and “prevention” refer to the prevention of the onset, recurrence, or spread of the disease in a patient resulting from the administration of a prophylactic or therapeutic agent.

The term “effective amount” refers to an amount of a compound as described herein or other active ingredient sufficient to provide a therapeutic or prophylactic benefit in the treatment or prevention of a disease as described herein or to delay or minimize symptoms associated with the disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enbances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

The present disclosure provides, in various embodiments, compound of formula (1) or a pharmaceutically acceptable salt thereof:

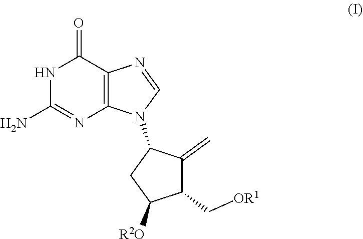

wherein
R¹ is H or is X-Lₘ,
m=1 or 2;
X-Lₘ is —C(═O)L, —C(═O)OL, —C(═O)NH(L), —C(═O)N(L)₂, —CH(R)OC(═O)L, —C(═O)CH(R)—NH(L), —C(═O)CH(R)—N(L)₂, —P(═O)(NHL)₂, —P(═O)(NHL)(NL₂), or —P(═O)(NL₂)₂,
each L is independently selected from (C1-22, linear and branched)alkyl, (C3-22, linear and branched)alkenyl, (C3-7)cycloalkyl, (CHR)ₙ-phenyl wherein n=0 or 1, and —CHR—N(R)₂;
or R¹ is —OCH(R)OP(═O)(OH)₂, a phosphate residue or its derivative residue comprising a monophosphate, a diphosphate, a triphosphate, a phosphonate, a phosphate polyester, a phosphate monoamidate, a phosphate diamidate, a phosphorothioate, a phosphoroselenoate, or a phosphoroboranoate;
R is H, (C1-22)alkyl, (C3-22)alkenyl, (C3-7)cycloalkyl, or (C6—C12)aryl;
R²is H or is X-Lₘ;
or R² is —OCH(R)OP(═O)(OH)₂, a phosphate residue or its derivative residue comprising a monophosphate, a diphosphate, a triphosphate, a phosphonate, a phosphate polyester, a phosphate monoamidate, a phosphate diamidate, a phosphorothioatc, a phosphoroselenoate, or a phosphoroboranoate,
and wherein R¹ and R² are not simultaneously H.

In various embodiments, each of R¹ and R² is X-Lₘ. In some embodiments, one or both instances of X-Lₘ is —C(═O)L. In exemplary embodiments, cach of each of R¹ and R² is —C(═O)L.

In other embodiments, notwithstanding the provisions of formula (I) as defined herein, formula (I) excludes one or more of the following combinations:
a) R¹=H and R²=X-Lₘ, R¹=X-Lₘ and R²=H, or R¹=R²=X-Lₘ. In these combinations, X-Lm is —C(═O)L, —C(═O)OL, or —C(═O)NH(L), and each L is independently selected from (C6-30, linear and branched)alkyl, (C7-30, linear and branched)alkenyl wherein the alkenyl can comprise 1-6 unsaturations;
b) R¹=—C(═O)CH₂(CH₂)ₙ—CH₃, n=3, 5, 7, 9, 11, 13, 15, and R²=H; and
c) R¹ is a monophosphate, and R² is selected from the group consisting of hydrogen, substituted or unsubstituted C₁-C₁₀ alkyl, substituted or unsubstituted C₃-C₁₀ cycloalkyl, substituted or unsubstituted C₂-C₁₂ alkanoyl, substituted or unsubstituted —C(O)O—C₁-C₁₀ alkyl; wherein substituents selected from the group consisting of halogen, C₁-C₃ alkyl, C₁-C₃ haloalkyl, nitro, hydroxy, —NR^{a}R^{b}, and a cyano group, wherein each of R^{a} and R^{b} is independently H, C₁-C₃ alkyl, C₃-C₆ cycloalkyl, or C-C₃ haloalkyl.

In additional embodiments, the present disclosure provides compounds 1-38, and pharmaceutically acceptable salts thereof, as shown in Table 1 below.

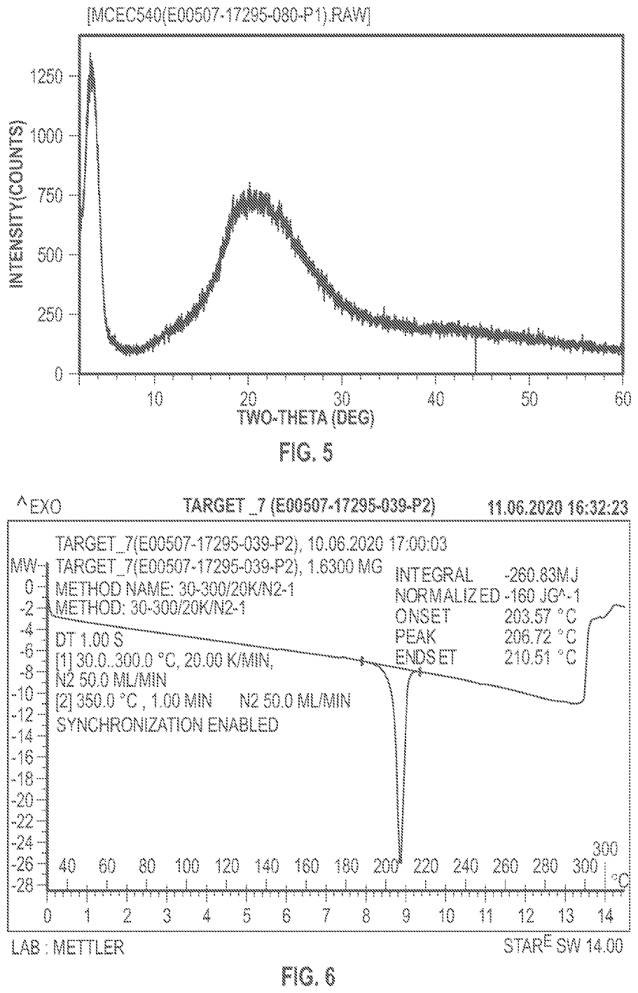

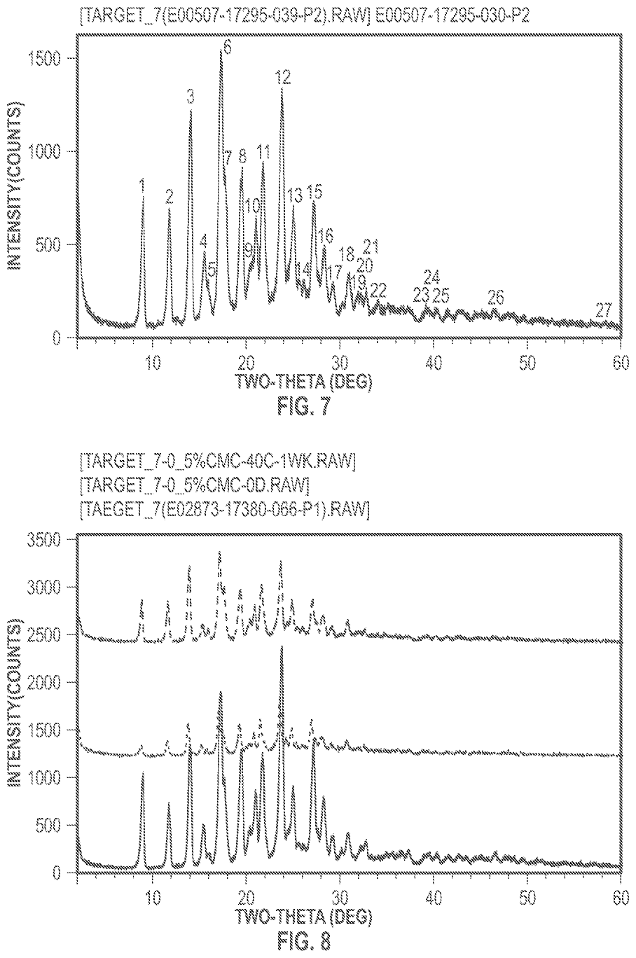

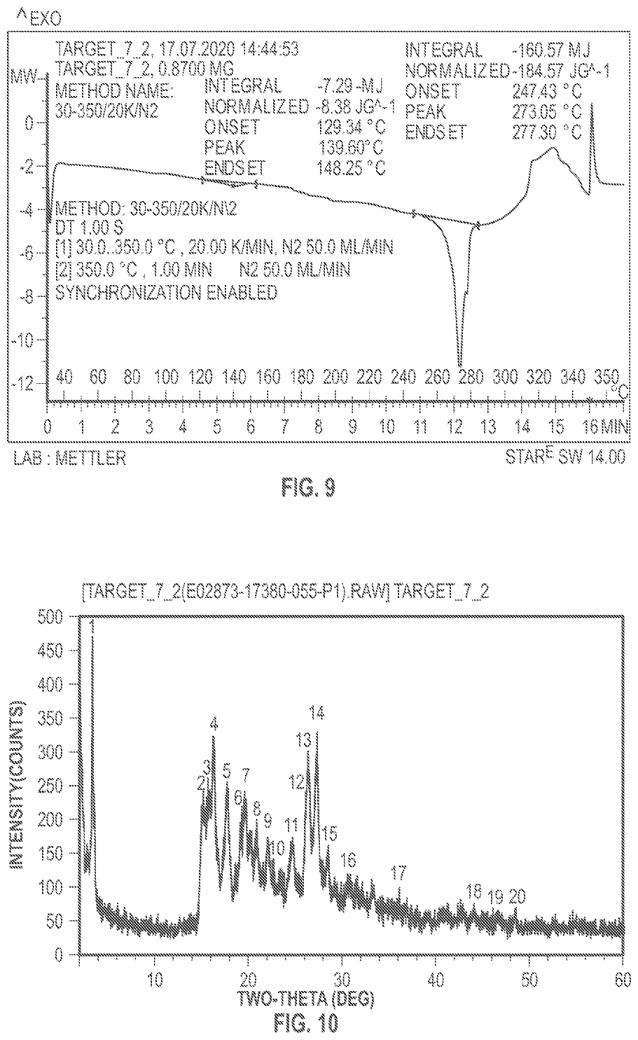

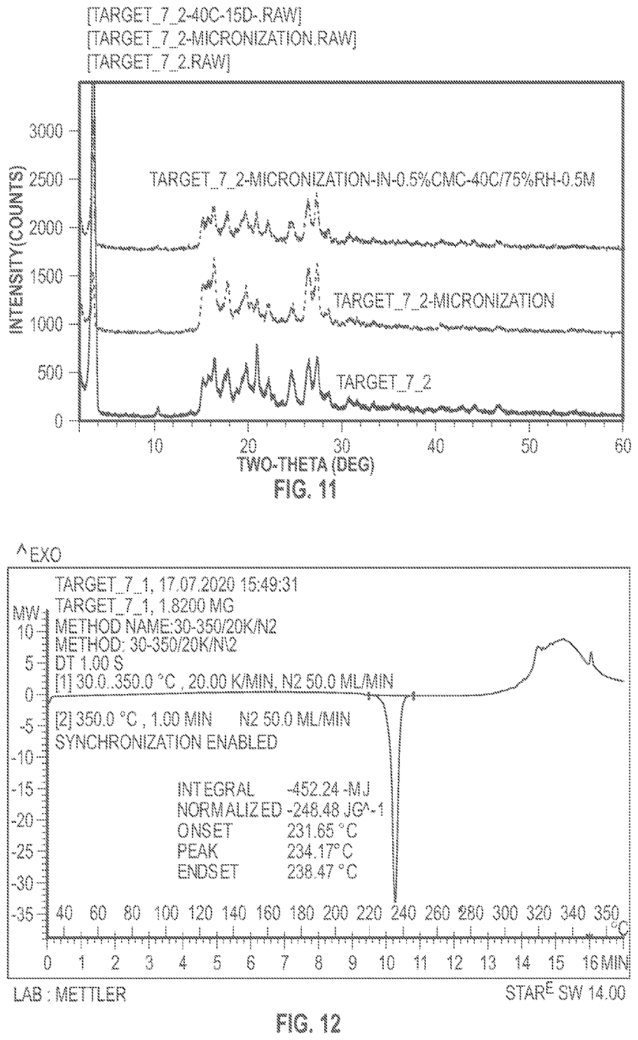

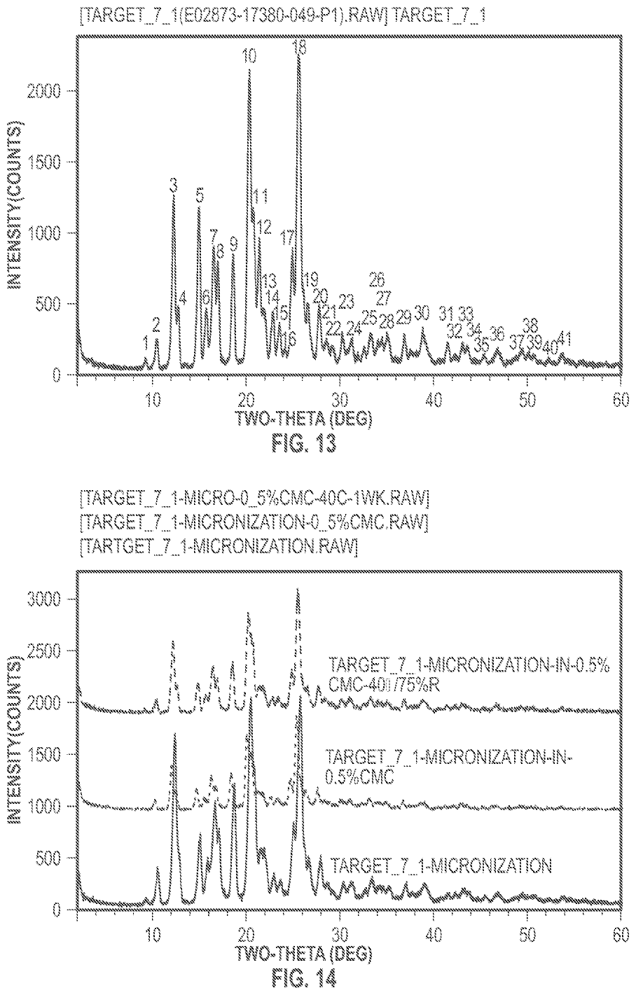

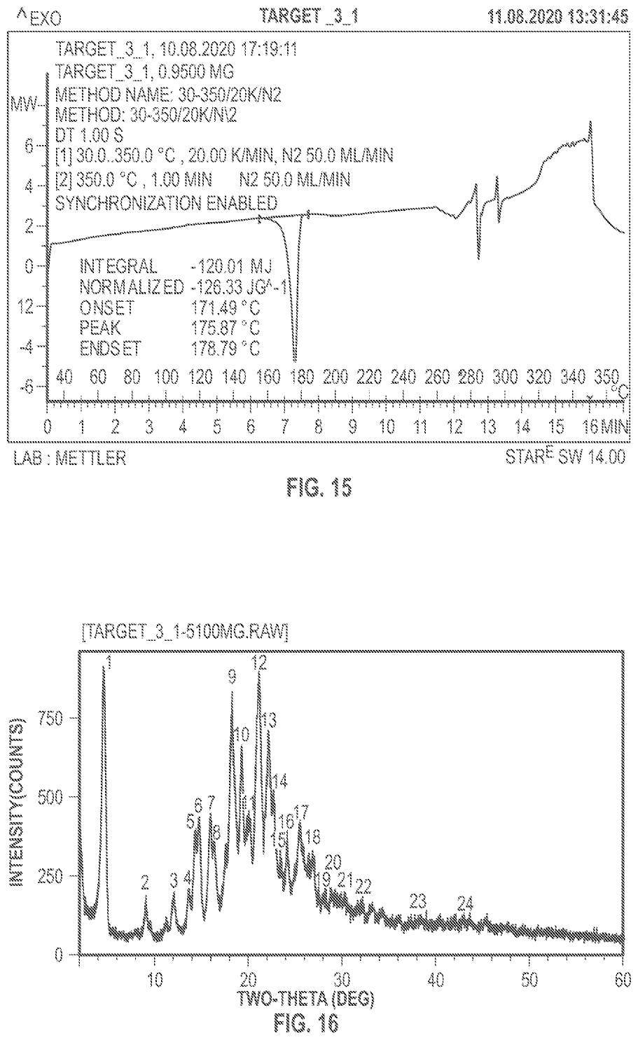

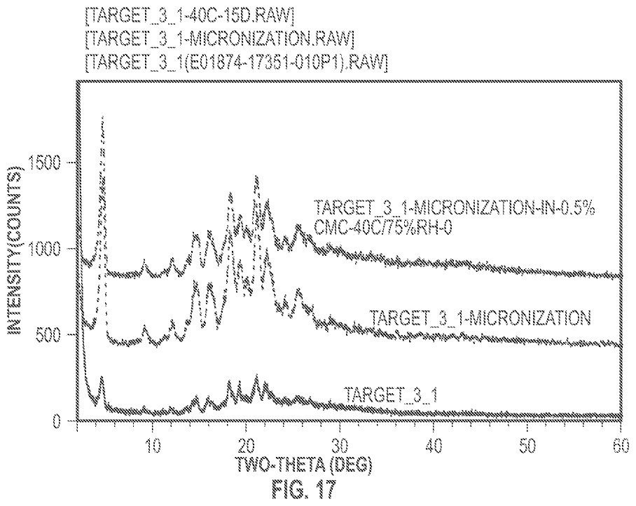

In various embodiments, the compound or pharmaceutically acceptable salt thereof as described herein is solid. For example, the solid is chosen from an amorphous solid and one or more of crystalline solids (polymorphs). Amorphous and crystalline solids are characterized, for example, by instrumental techniques known in the art, including X-ray powder diffraction, and differential scanning calorimetry (DSC). Exemplary characterizing data of crystalline solids of the present compounds include those presented in FIGS. 4-14.

In an illustrative embodiment, the present disclosure provides compound 11 in crystalline form:

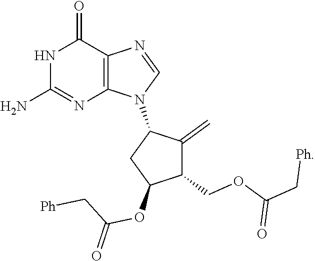 [SMILES: C=C1[C@H](COC(=O)Cc2ccccc2)[C@@H](OC(=O)Cc2ccccc2)C[C@@H]1n1cnc2c(=O)[nH]c(N)nc21]

The solid crystalline form of compound 11 is characterized by an X-ray powder diffractogram comprising the following peaks: 9.00, 17.28, 21.72, and 23.80°2θ±0.20°2θ as determined on a diffractometer using Cu—Kα₁ radiation at a wavelength of 1.54056 Å. In a further embodiment, the diffractogram further comprises the following peaks: 11.74, 19.48, 25.00, and 27.16°2θ±0.20°2θ.

In another illustrative embodiment, the present disclosure provides compound 36 in crystalline form:

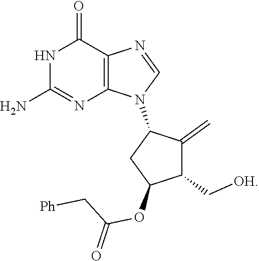 [SMILES: C=C1[C@H](CO)[C@@H](OC(=O)Cc2ccccc2)C[C@@H]1n1cnc2c(=O)[nH]c(N)nc21]

The solid crystalline form of compound 36 is characterized by an X-ray powder diffractogram comprising the following peaks: 16.44, 19.36, 20.88, and 26.46°2θ±0.20°2θ as determined on a diffractometer using Cu—Kα₁ radiation at a wavelength of 1.54056 Å. In an embodiment, the diffractogram further comprises the following peaks: 15.18, 22.16, 24.56, and 28.58°2θ±0.2θ°2θ.

The present disclosure also provides compound 29 in crystalline form:

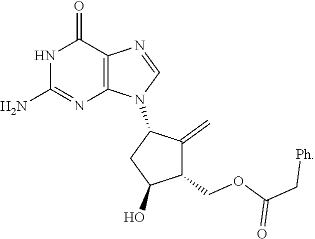 [SMILES: C=C1[C@H](COC(=O)Cc2ccccc2)[C@@H](O)C[C@@H]1n1cnc2c(=O)[nH]c(N)nc21]

The solid crystalline form of compound 29 is characterized by an X-ray powder diffractogram comprising the following peaks: 12.30, 18.62, 20.34, and 25.54°2θ±0.20°2θ as determined on a difractometer using Cu—Kα₁ radiation at a wavelength of 1.54056 Å. In an embodiment, the diffractogram further comprises the following peaks: 14.96, 16.54, 21.38, and 27.74°2θ±0.2°2θ.

In further illustrative embodiments, the present disclosure provides a compound or pharmaceutically acceptable salt thereof as shown in Table 1. The compounds are disclosed with characterizing data (NMR and LCMS).

TABLE 1
Exemplary Compounds 1-38

| Cpd | | |
|---|---|---|
| # | Structure | Characterization Data (NMR and LCMS) |
| 1 | 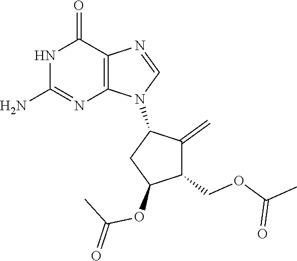 [SMILES: C=C1[C@H](COC(C)=O)[C@@H](OC(C)=O)C[C@@H]1n1cnc2c(=O)[nH]c(N)nc21] | MS-ESI: m/z 362.42 observed (M + H)⁺ |
| 2 | 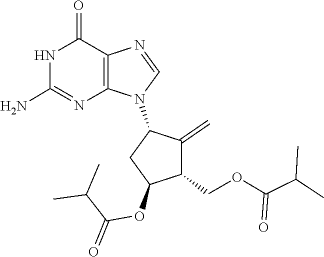 [SMILES: C=C1[C@H](COC(=O)C(C)C)[C@@H](OC(=O)C(C)C)C[C@@H]1n1cnc2c(=O)[nH]c(N)nc21] | ¹H NMR (400 MHz, DMSO-d₆) δ 10.62 (s, 1H), 7.74 (s, 1H), 6.41 (br s, 2H), 5.37-5.33 (m, 1H), 5.21-5.20 (m, 2H), 4.66 (t, J = 2.4 Hz, 1H), 4.26 (d, J = 7.2 Hz, 2H), 2.94-2.89 (m, 1H), 2.69-2.50 (m, 3H), 2.27-2.22 (m, 1H), 1.13-1.09 (m, 12H). MS-ESI: m/z 418.2 observed (M + H)⁺ |
| 3 | 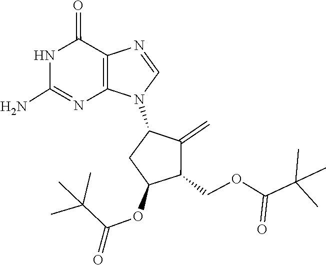 [SMILES: C=C1[C@H](COC(=O)C(C)(C)C)[C@@H](OC(=O)C(C)(C)C)C[C@@H]1n1cnc2c(=O)[nH]c(N)nc21] | ¹H NMR (400 MHz, DMSO-d₆) δ 10.61 (s, 1H), 7.74 (s, 1H), 6.39 (br s, 2H), 5.34 (t, J = 8.0 Hz, 1H), 5.21-5.18 (m, 2H), 4.65 (t, J = 1.6 Hz, 1H), 4.25 (d, J = 7.6 Hz, 2H), 2.91 (t, J = 6.8 Hz, 1H), 2.73-2.65 (m, 1H), 2.26- 2.21 (m, 1H), 1.19 (s, 9H), 1.18 (s, 9H). MS-ESI: m/z 446.2 observed (M + H)⁺ |
| 4 | 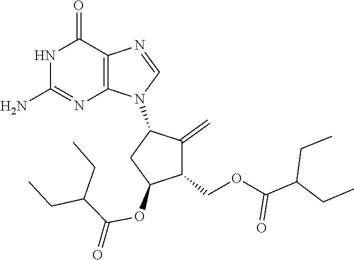 [SMILES: C=C1[C@H](COC(=O)C(CC)CC)[C@@H](OC(=O)C(CC)CC)C[C@@H]1n1cnc2c(=O)[nH]c(N)nc21] | ¹H NMR (400 MHz, DMSO-d₆) δ 10.60 (s, 1H), 7.73 (s, 1H), 6.39 (br s, 2H), 5.33 (t, J = 8.0 Hz, 1H), 5.25 (s, 2H), 4.69 (t, J = 2.4 Hz, 1H), 4.29 (d, J = 7.2 Hz, 2H), 2.98-2.90 (m, 1H), 2.72-2.63 (m, 1H), 2.28-2.16 (m, 3H), 1.60-1.45 (m, 8H), 0.87-0.82 (m, 12H). MS-ESI: m/z 474.2 observed (M + H)⁺ |
| 5 | 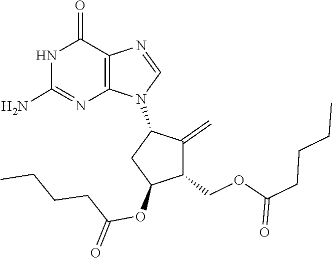 [SMILES: C=C1[C@H](COC(=O)CCCC)[C@@H](OC(=O)CCCC)C[C@@H]1n1cnc2c(=O)[nH]c(N)nc21] | ¹H NMR (400 MHz, DMSO-d₆) δ 10.61 (s, 1H), 7.73 (s, 1H), 6.41 (br s, 2H), 5.33 (t, J = 8.8 Hz, 1H), 5.20-5.18 (m, 2H), 4.65 (s, 1H), 4.26 (d, J = 6.4 Hz, 2H), 2.99-2.88 (m, 1H), 2.66-2.58 (m, 1H), 2.37-2.29 (m, 4H), 2.26-2.21 (m, 1H), 1.55-1.49 (m, 4H), 1.34- 1.23 (m, 4H), 0.87 (t, J = 7.6 Hz, 3H). MS-ESI: m/z 446.2 observed (M + H)⁺ |
| 6 | 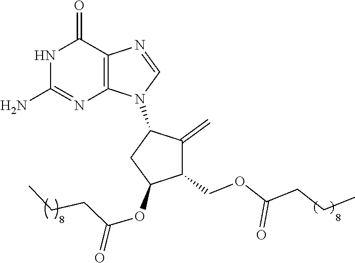 | ¹H NMR (400 MHz, DMSO-d₆) δ 10.59 (s, 1H), 7.72 (s, 1H), 6.40 (s, 2H), 5.35-7.30 (m, 1H), 5.19 (s, 2H), 4.64 (s, 1H), 4.26- 4.23 (m, 2H), 2.93-2.90 (m, 1H), 2.64-2.58 (m, 1H), 2.35-2.27 (m, 4H), 2.25-2.21 (m, 1H), 1.53-1.50 (m, 4H), 1.23 (s, 28H), 0.86- 0.83 (m, 6H). MS-ESI: m/z 614.5 observed (M + H)⁺ |
| 7 | 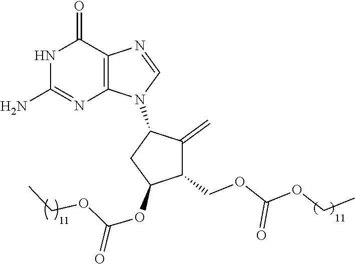 | MS-ESI: m/z 702.5 observed (M + H)⁺ |
| 8 | 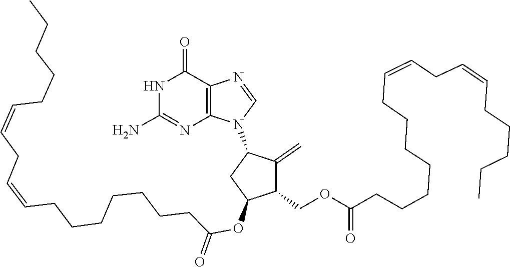 [SMILES: C=C1[C@H](COC(=O)CCCCCCC/C=C\C/C=C\CCCCC)[C@@H](OC(=O)CCCCCCC/C=C\C/C=C\CCCCC)C[C@@H]1n1cnc2c(=O)[nH]c(N)nc21] | ¹H NMR (400 MHz, DMSO-d₆) δ 10.61 (s, 1H), 7.72 (s, 1H), 6.41 (br s, 2H), 5.37-5.19 (m, 9H), 5.15 (s, 2H), 4.64 (s, 1H), 4.30- 4.20 (m, 2H), 2.94-2.88 (m, 1H), 2.74-2.72 (m, 4H), 2.66-2.58 (m, 1H), 2.35-2.27 (m, 4H), 2.25-2.19 (m, 1H), 2.03-1.98 (m, 8H), 1.58-1.47 (m, 4H), 1.32-1.26 (m, 24H), 0.85 (t, J = 7.2 Hz, 6H). MS-ESI: m/z 802.6 observed (M + H)⁺ |
| 9 | 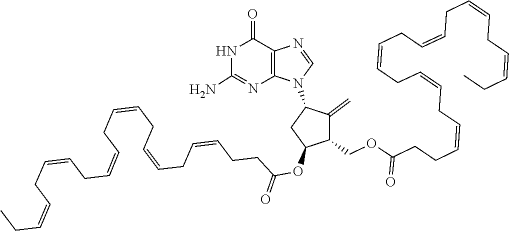 [SMILES: C=C1[C@H](COC(=O)CC/C=C\C/C=C\C/C=C\C/C=C\C/C=C\C/C=C\CC)[C@@H](OC(=O)CC/C=C\C/C=C\C/C=C\C/C=C\C/C=C\C/C=C\CC)C[C@@H]1n1cnc2c(=O)[nH]c(N)nc21] | ¹H NMR (400 MHz, DMSO-d₆) δ 10.63 (s, 1H), 7.72 (s, 1H), 6.41 (br s, 2H), 5.36-5.26 (m, 25H), 5.20 (s, 2H), 4.65 (s, 1H), 4.26 (d, J = 6.8 Hz, 2H), 2.98-2.92 (m, 1H), 2.89- 2.76 (m, 20H), 2.68-2.58 (m, 1H), 2.41-2.20 (m, 9H), 2.20-2.00 (m, 4H), 0.91 (t, J = 7.6 Hz, 6H). MS-ESI: m/z 898.6 observed (M + H)⁺ |
| 10 | 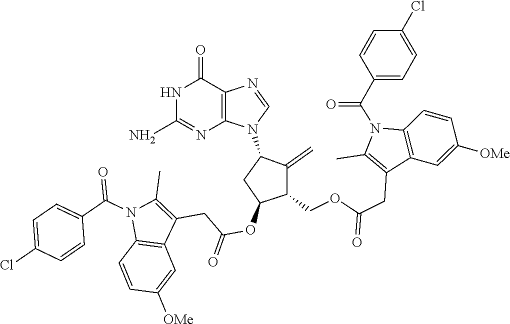 [SMILES: C=C1[C@H](COC(=O)Cc2c(C)n(C(=O)c3ccc(Cl)cc3)c3ccc(OC)cc23)[C@@H](OC(=O)Cc2c(C)n(C(=O)c3ccc(Cl)cc3)c3ccc(OC)cc23)C[C@@H]1n1cnc2c(=O)[nH]c(N)nc21] | ¹H NMR (400 MHz, DMSO-d₆) δ 10.62 (s, 1H), 7.68-7.58 (m, 9H), 7.03-7.01 (m, 2H), 6.93-6.88 (m, 2H), 6.72 - 6.67 (m, 2H), 6.37 (s, 2H), 5.33 (t, J = 8.8 Hz, 1H), 5.17 (s, 2H), 4.61 (s, 1H), 4.31 (d, J = 6.8 Hz, 1H), 3.80 (s, 4H), 3.73 (s, 1H), 3.69 (s, 3H), 3.01-2.95 (m, 1H), 2.55-2.50 (m, 1H), 2.26- 2.22 (m, 4H), 2.15 (s, 3H). MS-ESI: m/z 956.2 observed (M + H)⁺ |
| 11 | 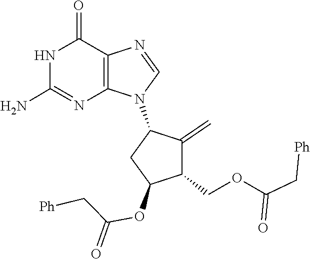 [SMILES: C=C1[C@H](COC(=O)Cc2ccccc2)[C@@H](OC(=O)Cc2ccccc2)C[C@@H]1n1cnc2c(=O)[nH]c(N)nc21] | ¹H NMR (400 MHz, DMSO-d₆) δ 10.64 (s, 1H), 7.67 (s, 1H), 7.37-7.22 (m, 10H), 6.44 (br s, 2H), 5.33 (t, J = 8.4 Hz, 1H), 5.20 (d, J = 4.8 Hz, 1H), 5.16 (s, 1H), 4.61 (s, 1H), 4.28 (d, J = 7.2 Hz, 2H), 3.71 (s, 4H), 3.00- 2.90 (m, 1H), 2.59-2.51 (m, 1H), 2.27-2.22 (m, 1H). MS-ESI: m/z 514.2 observed (M + H)⁺ |
| 12 | 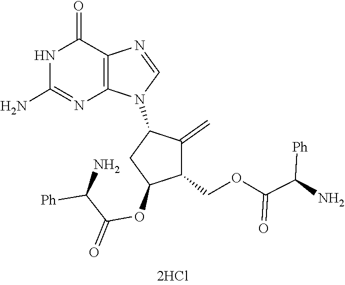 [SMILES: C=C1[C@H](COC(=O)[C@H](N)c2ccccc2)[C@@H](OC(=O)[C@H](N)c2ccccc2)C[C@@H]1n1cnc2c(=O)[nH]c(N)nc21] | ¹H NMR (400 MHz, DMSO-d₆) δ 11.74 (s, 1H), 9.31-9.26 (m, 6H), 8.88-8.78 (m, 1H), 7.57-7.39 (m, 10H), 7.25 (s, 2H), 5.54-5.05 (m, 5H), 5.03-4.79 (m, 1H), 4.73-4.27 (m, 2H), 3.12 (s, 0.5H), 2.87 (s, 0.5H), 2.73- 2.55 (m, 1H), 2.51-2.14 (m, 1H). MS-ESI: m/z 544.2 observed (M + H)⁺ |
| 13 | 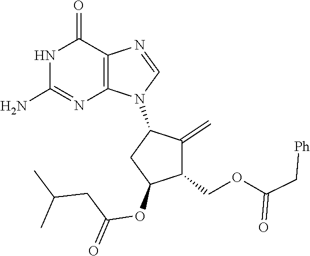 | ¹H NMR (400 MHz, DMSO-d₆) δ 10.59 (s, 1H), 7.67 (s, 1H), 7.32-7.28 (m, 5H), 6.40 (s, 1H), 5.34-5.29 (m, 1H), 5.19-5.16 (m, 2H), 4.61 (s, 1H), 4.30 (d, J = 6.8 Hz, 2H), 3.74 (s, 2H), 2.97-2.93 (m, 1H), 2.65-2.53 (m, 1H), 2.22-2.20 (m, 3H), 2.05-1.97 (m, 1H), 0.92 (d, J = 7.2 Hz, 6H). MS-ESI: m/z 580.2 observed (M + H)⁺ |
| 14 | 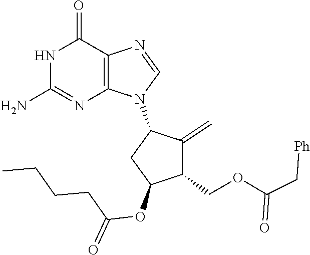 [SMILES: C=C1[C@H](COC(=O)Cc2ccccc2)[C@@H](OC(=O)CCCC)C[C@@H]1n1cnc2c(=O)[nH]c(N)nc21] | ¹H NMR (400 MHz, DMSO-d₆) δ 10.59 (s, 1H), 7.67 (s, 1H), 7.33-7.23 (m, 5H), 6.40 (br s, 2H), 5.34-5.27 (m, 1H), 5.17-5.15 (m, 2H), 4.60 (s, 1H), 4.28 (d, J = 7.6 Hz, 2H), 3.73 (s, 2H), 2.96-2.87 (m, 1H), 2.59-2.49 (m, 1H), 2.31 (t, J = 7.6 Hz, 2H), 2.24-2.18 (m, 1H), 1.56-1.47 (m, 2H), 1.33-1.23 (m, 2H), 0.86 (t, J = 7.6 Hz, 3H). MS-ESI: m/z 480.2 observed (M + H)⁺ |
| 15 | 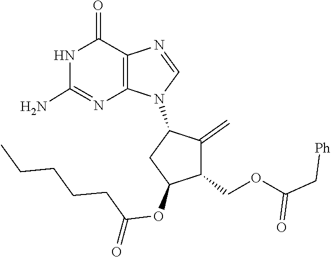 [SMILES: C=C1[C@H](COC(=O)Cc2ccccc2)[C@@H](OC(=O)CCCCC)C[C@@H]1n1cnc2c(=O)[nH]c(N)nc21] | ¹H NMR (400 MHz, DMSO-d₆) δ 10.72 (s, 1H), 7.79-7.76 (m, 1H), 7.46-7.37 (m, 5H), 6.53 (br s, 2H), 5.43-5.40 (m, 1H), 5.28 (s, 2H), 4.72 (s, 1H), 4.41 (d, J = 6.9 Hz, 2H), 3.85 (s, 2H), 3.04 (s, 1H), 2.70-2.61 (m, 1H), 2.43 (t, J = 7.5 Hz, 2H), 2.38-2.30 (m, 1H), 1.68-1.64 (m, 2H), 1.40-1.38 (m, 4H), 0.99 (t, J = 6.3 Hz, 3H). MS-ESI: m/z 493.2 observed (M + H)⁺ |
| 16 | 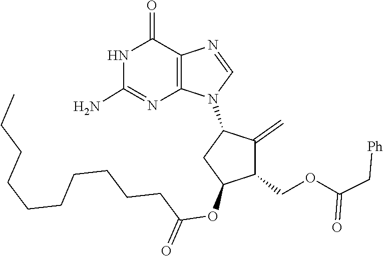 | ¹H NMR (400 MHz, DMSO-d₆) δ 10.60 (s, 1H), 7.62 (s, 1H), 7.33-7.22 (m, 5H), 6.42 (s, 1H), 5.34-5.29 (m, 1H), 5,18-5.15 (m, 2H), 4.60 (s, 1H), 4.28 (d, J = 7.6 Hz, 2H), 3.73 (s, 2H), 2.96 -2.89 (m, 1H), 2.60-2.52 (m, 1H), 2.30 (t, J = 7.6 Hz, 2H), 2.23-2.18 (m, 1H), 1.57-1.49 (m, 2H), 1.25-1.23 (m, 14H), 0.84 (t, J = 7.2 Hz, 3H). MS-ESI: m/z 563.3 observed (M + H)⁺ |
| 17 | 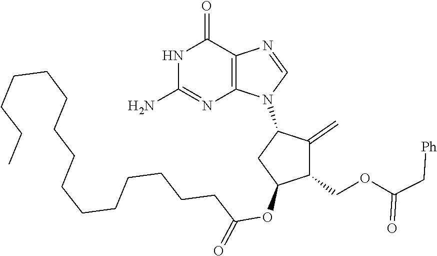 [SMILES: C=C1[C@H](COC(=O)Cc2ccccc2)[C@@H](OC(=O)CCCCCCCCCCCCCCC)C[C@@H]1n1cnc2c(=O)[nH]c(N)nc21] | ¹H NMR (400 MHz, DMSO-d₆) δ 10.59 (s, 1H), 7.67 (s, 1H), 7.33-7.21 (m, 5H), 6.40 (br s, 2H), 5.33-5.29 (m, 1H), 5.17-5.14 (m, 2H), 4.59 (s, 1H), 4.28 (d, J = 7.2 Hz, 2H), 3.73 (s, 2H), 2.97-2.86 (m, 1H), 2.59-2.54 (m, 1H), 2.30 (t, J = 7.6 Hz, 2H), 2.23-2.15 (m, 1H), 1.58-1.47 (m, 2H), 1.24-1.22 (m, 24H), 0.85 (t, J = 6.8 Hz, 3H). MS-ESI: m/z 633.4 observed (M + H)⁺ |
| 18 | 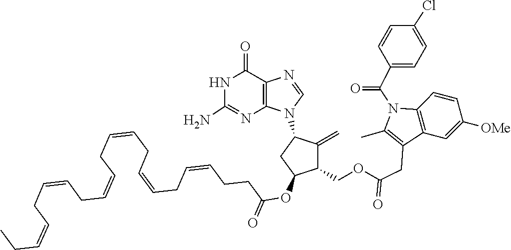 [SMILES: C=C1[C@H](COC(=O)Cc2c(C)n(C(=O)c3ccc(Cl)cc3)c3ccc(OC)cc23)[C@@H](OC(=O)CC/C=C\C/C=C\C/C=C\C/C=C\C/C=C\C/C=C\CC)C[C@@H]1n1cnc2c(=O)[nH]c(N)nc21] | ¹H NMR (400 MHz, DMSO-d₆) δ 10.58 (s, 1H), 7.68-7.61 (m, 5H), 7.04 (d, J = 2.0 Hz, 1H), 6.91 (d, J = 8.8 Hz, 1H), 6.70 (dd, J = 8.8, 2.4 Hz, 1H), 6.39 (br s, 2H), 5.37-5.31 (m, 13H), 5.15-5.12 (m, 2H), 4.59 (s, 1H), 4.30 (d, J = 6.4 Hz, 2H), 3.84 (s, 2H), 3.74 (s, 3H), 2.96-2.89 (m, 1H), 2.80-2.74 (m, 10H), 2.50-2.46 (m, 1H), 2.37-2.26 (m, 4H), 2.23 (s, 3H), 2.21-2.13 (m, 1H), 2.07-1.95 (m, 2H), 0.90 (t, J = 7.6 Hz, 3H), MS-ESI: m/z 927.4 observed (M + H)⁺ |
| 19 | 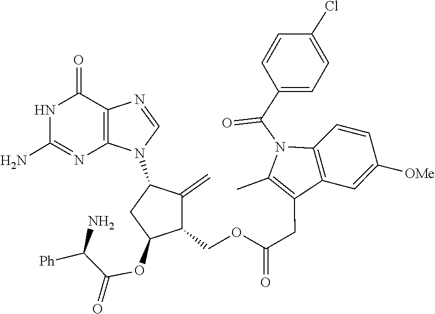 [SMILES: C=C1[C@H](COC(=O)Cc2c(C)n(C(=O)c3ccc(Cl)cc3)c3ccc(OC)cc23)[C@@H](OC(=O)[C@H](N)c2ccccc2)C[C@@H]1n1cnc2c(=O)[nH]c(N)nc21] | ¹H NMR (400 MHz, DMSO-d₆) δ 10.64 (s, 1H), 7.68-7.56 (m, 5H), 7.38-7.21 (m, 5H), 7.04 (t, J = 2.8 Hz, 1H), 6.96-6.92 (m, 1H), 6.76-6.72 (m, 1H), 6.39 (d, J = 8.4 Hz, 2H), 5.32-5.08 (m, 3H), 4.63-4.56 (m, 1H), 4.52 (d, J = 4.8 Hz, 1H), 4.32-4.20 (m, 2H), 3.84- 3.76 (m, 2H), 3.72 (t, J = 4.4 Hz, 3H), 3.00- 2.93 (m, 0.5H), 2.79-2.73 (m, 0.5H), 2.56- 2.42 (m, 1H), 2.40-2.26 (m, 2.5H), 2.20 (d, J = 13.6 Hz, 3H), 2.02-1.96 (m, 0.5H). MS-ESI: m/z 750.2 observed (M + H)⁺ |
| 20 | 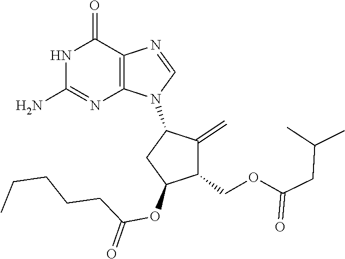 [SMILES: C=C1[C@H](COC(=O)CC(C)C)[C@@H](OC(=O)CCCCC)C[C@@H]1n1cnc2c(=O)[nH]c(N)nc21] | ¹H NMR (400 MHz, DMSO-d₆) δ 10.63 (s, 1H), 7.75 (s, 1H), 6.44 (br s, 2H), 5.39-5.32 (m, 1H), 5.24-5.22 (m, 2H), 4.69-4.65 (m, 1H), 4.29 (d, J = 6.9 Hz, 2H), 3.00-2.89 (m, 1H), 2.69-2.52 (m, 1H), 2.35-2.25 (m, 5H), 2.07-1.98 (m, 1H), 1.61-1.51 (m, 2H), 1.33- 1.26 (m, 4H), 0.95 (s, 3H), 0.93 (s, 3H), 0.91-0.86 (m, 3H). MS-ESI: m/z 460.2 observed (M + H)⁺ |
| 21 | 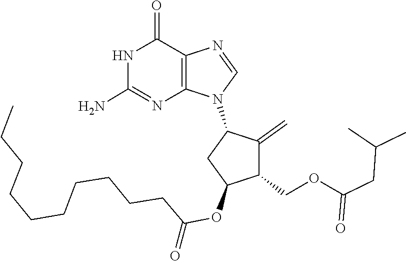 [SMILES: C=C1[C@H](COC(=O)CC(C)C)[C@@H](OC(=O)CCCCCCCCCC)C[C@@H]1n1cnc2c(=O)[nH]c(N)nc21] | ¹H NMR (400 MHz, DMSO-d₆) δ 10.59 (s, 1H), 7.72 (s, 1H), 6.41 (s, 2H), 5.35-5.30 (m, 1H), 5.21-5.18 (m, 2H), 4.65 (s, 1H), 4.27-4.25 (m, 2H), 2.96 -2.89 (m, 1H), 2.67- 2.57 (m, 1H), 2.30 (t, J = 7.6 Hz, 2H), 2.25- 2.23 (m, 3H), 1.99-1.97 (m, 1H), 1.56-1.48 (m, 2H), 1.25-1.23 (m, 14H), 0.92-0.90 (m, 6H), 0.84 (t, J = 7.2 Hz, 3H). MS-ESI: m/z 530.2 observed (M + H)⁺ |
| 22 | 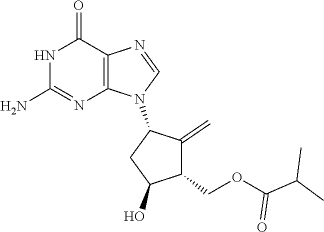 [SMILES: C=C1[C@H](COC(=O)C(C)C)[C@@H](O)C[C@@H]1n1cnc2c(=O)[nH]c(N)nc21] | ¹H NMR (400 MHz, DMSO-d₆) δ 10.57 (s, 1H), 7.67 (s, 1H), 6.39 (br s, 2H), 5.38 (t, J = 8.4 Hz, 1H), 5.15 (t, J = 3.2 Hz, 1H), 5.08 (d, J = 3.2 Hz, 1H), 4.61 (t, J = 2.0 Hz, 1H), 4.22-4.14 (m, 3H), 2.79-2.70 (m, 1H), 2.59- 2.50 (m, 1H), 2.35-2.28 (m, 1H), 2.12-2.05 (m, 1H), 1.12-1.09 (m, 6H). MS-ESI: m/z 348.1 observed (M + H)⁺ |
| 23 | 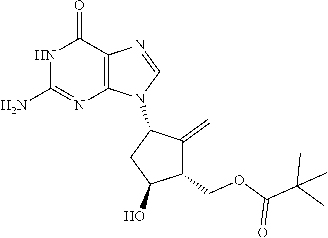 [SMILES: C=C1[C@H](COC(=O)C(C)(C)C)[C@@H](O)C[C@@H]1n1cnc2c(=O)[nH]c(N)nc21] | ¹H NMR (400 MHz, DMSO-d₆) δ 10.56 (s, 1H), 7.66 (s, 1H), 6.39 (br s, 2H), 5.38 (t, J = 8.0 Hz, 1H), 5.14 (s, 1H), 5.09 (d, J = 3.2 Hz, 1H), 4.61 (s, 1H), 4.22-4.13 (m, 3H), 2.79-2.70 (m, 1H), 2.35-2.26 (m, 1H), 2.11- 2.04 (m, 1H), 1.17 (s, 9H). MS-ESI: m/z 362.1 observed (M + H)⁺ |
| 24 | 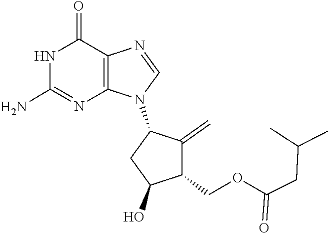 [SMILES: C=C1[C@H](COC(=O)CC(C)C)[C@@H](O)C[C@@H]1n1cnc2c(=O)[nH]c(N)nc21] | ¹H NMR (400 MHz, DMSO-d₆) δ 7.67 (s, 1H), 6.40 (s, 2H), 5.40-5.35 (m, 1H), 5.18- 5.16 (m, 1H), 5.11-5.07 (m, 1H), 4.62-4.61 (m, 1H), 4.23-4.16 (m, 3H), 2.77-2.70 (m, 1H), 2.34-2.27 (m, 1H), 2.22 (d, J = 7.2 Hz, 1H), 2.09-1.97 (m, 1H), 0.93-0.91 (m, 6H). MS-ESI: m/z 362.2 observed (M + H)⁺ |
| 25 | 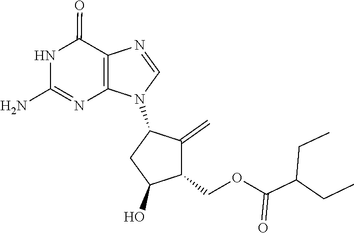 [SMILES: C=C1[C@H](COC(=O)C(CC)CC)[C@@H](O)C[C@@H]1n1cnc2c(=O)[nH]c(N)nc21] | ¹H NMR (400 MHz, DMSO-d₆) δ 10.57 (s, 1H), 7.66 (s, 1H), 6.39 (br s, 2H), 5.38 (t, J = 8.4 Hz, 1H), 5.16 (t, J = 2.4 Hz, 1H), 5.08 (d, J = 2.4 Hz, 1H), 4.62 (t, J = 2.0 Hz, 1H), 4.26-4.16 (m, 3H), 2.78-2.70 (m, 1H), 2.35- 2.20 (m, 2H), 2.11-2.05 (m, 1H), 1.58-1.46 (m, 4H), 0.84 (t, J = 7.2 Hz, 6H). MS-ESI: m/z 376.2 observed (M + H)⁺ |
| 26 | 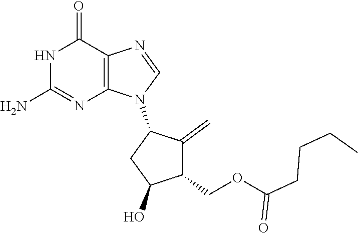 [SMILES: C=C1[C@H](COC(=O)CCCC)[C@@H](O)C[C@@H]1n1cnc2c(=O)[nH]c(N)nc21] | ¹H NMR (400 MHz, DMSO-d₆) δ 10.60 (s, 1H), 7.67 (s, 1H), 6.41 (br s, 2H), 5.38 (t, J = 8.8 Hz, 1H), 5.15 (s, 1H), 5.08 (d, J = 2.4 Hz, 1H), 4.62 (s, 1H), 4.22-4.17 (m, 3H), 2.78-2.68 (m, 1H), 2.36-2.29 (m, 3H), 2.10- 2.05 (m, 1H), 1.57-1.49 (m, 2H), 1.35-1.23 (m, 2H), 0.87 (t, J = 7.6 Hz, 3H). MS-ESI: m/z 362.2 observed (M + H)⁺ |
| 27 | 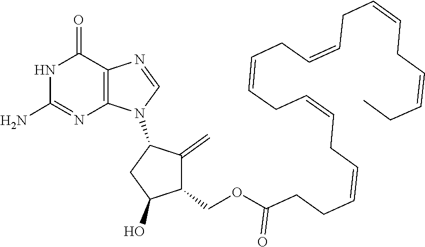 [SMILES: C=C1[C@H](COC(=O)CC/C=C\C/C=C\C/C=C\C/C=C\C/C=C\C/C=C\CC)[C@@H](O)C[C@@H]1n1cnc2c(=O)[nH]c(N)nc21] | ¹H NMR (400 MHz, DMSO-d₆) δ 10.58 (s, 1H), 7.67 (s, 1H), 6.40 (br s, 2H), 5.35-5.34 (m, 13H), 5.15 (s, 1H), 5.08 (s, 1H), 4.61 (s, 1H), 4.21-4.12 (m, 3H), 2.87-2.70 (m, 11H), 2.51-2.49 (m, 1H), 2.45-2.36 (m, 2H), 2.35- 2.24 (m, 3H), 2.13-1.98 (m, 2H), 0.95-0.82 (m, 3H). MS-ESI: m/z 588.3 observed (M + H)⁺ |
| 28 | 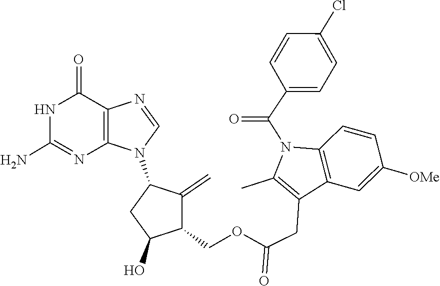 [SMILES: C=C1[C@H](COC(=O)Cc2c(C)n(C(=O)c3ccc(Cl)cc3)c3ccc(OC)cc23)[C@@H](O)C[C@@H]1n1cnc2c(=O)[nH]c(N)nc21] | ¹H NMR (400 MHz, DMSO-d₆) δ 10.56 (s, 1H), 7.68-7.62 (m, 4H), 7.57 (s, 1H), 7.03 (d, J = 1.8 Hz, 1H), 6.92 (d, J = 8.8 Hz, 1H), 6.72 (dd, J = 8.4, 2.8 Hz, 1H), 6.39 (br s, 2H), 5.36 (t, J = 9.6 Hz, 1H), 5.10 (s, 1H), 5.06 (d, J = 3.2 Hz, 1H), 4.56 (s, 1H), 4.25- 4.21 (m, 2H), 4.13 (s, 1H), 3.83 (s, 2H), 3.75 (s, 3H), 2.73 (s, 1H), 2.23 (s, 3H), 2.21- 2.15 (m, 1H), 2.05-1.99 (m, 1H). MS-ESI: m/z 617.1 observed (M + H)⁺ |
| 29 | 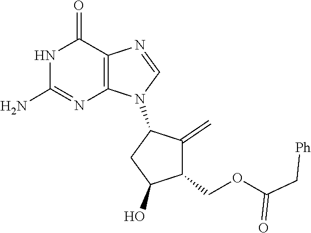 [SMILES: C=C1[C@H](COC(=O)Cc2ccccc2)[C@@H](O)C[C@@H]1n1cnc2c(=O)[nH]c(N)nc21] | ¹H NMR (400 MHz, DMSO-d₆) δ 10.58 (s, 1H), 7.63 (s, 1H), 7.39-7.22 (m, 5H), 6.39 (br s, 2H), 5.37 (t, J = 8.0 Hz, 1H), 5.10 (s, 1H), 5.07 (d, J = 2.4 Hz, 1H), 4.58 (s, 1H), 4.26-4.17 (m, 2H), 4.12 (s, 1H), 3.72 (s, 2H), 2.79-2.70 (m, 1H), 2.30-2.22 (m, 1H), 2.10-2.03 (m, 1H). MS-ESI: m/z 396.1 observed (M + H)⁺ |
| 30 | 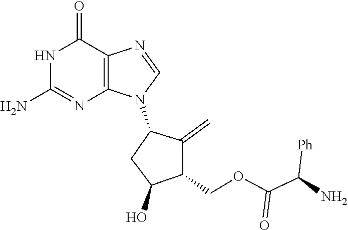 [SMILES: C=C1[C@H](COC(=O)[C@H](N)c2ccccc2)[C@@H](O)C[C@@H]1n1cnc2c(=O)[nH]c(N)nc21] | ¹H NMR (400 MHz, DMSO-d₆) δ 11.61 (s, 1H), 9.16 (s, 3H), 8.81 (d, J = 17.6 Hz, 1H), 7.56-7.43 (m, 5H), 7.17 (s, 2H), 5.49-5.42 (m, 1H), 5.36-5.31 (m, 1H), 5.07 (s, 1H), 4.70 (s, 1H), 4.42-4.27 (m, 2H), 4.14-4.06 (m, 1H), 2.80-2.75 (m, 1H), 2.38-2.22 (m, 1H), 2.04-1.95 (m, 1H). MS-ESI: m/z 411.2 observed (M + H)⁺ |
| 31 | 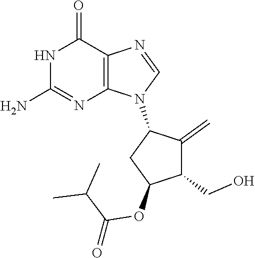 [SMILES: C=C1[C@H](CO)[C@@H](OC(=O)C(C)C)C[C@@H]1n1cnc2c(=O)[nH]c(N)nc21] | ¹H NMR (400 MHz, DMSO-d₆) δ 10.59 (s, 1H), 7.73 (s, 1H), 6.42 (br s, 2H), 5.33-5.26 (m, 2H), 5.17 (t, J = 2.0 Hz, 1H), 4.99 (s, 1H), 4.63 (t, J = 2.0 Hz, 1H), 3.63 (s, 2H), 2.68 (s, 1H), 2.57-2.48 (m, 2H), 2.20 (dd, J = 13.6, 8.0 Hz, 1H), 1.11-1.09 (m, 6H). MS-ESI: m/z 348.2 observed (M + H)⁺ |
| 32 | 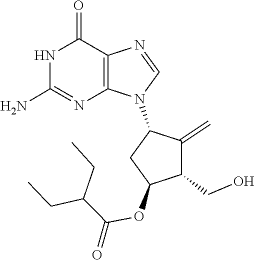 [SMILES: C=C1[C@H](CO)[C@@H](OC(=O)C(CC)CC)C[C@@H]1n1cnc2c(=O)[nH]c(N)nc21] | ¹H NMR (400 MHz, DMSO-d₆) 10.62 (s, 1H), 7.74 (s, 1H), 6.42 (br s, 2H), 5.31 (s, 2H), 5.20 (s, 1H), 5.02 (s, 1H), 4.66 (s, 1H), 3.65 (s, 2H), 2.69 (s, 1H), 2.59-2.52 (m, 1H), 2.19-2.17 (m, 2H), 1.54-1.48 (m, 4H), 0.86 (t, J = 6.8 Hz, 6H). MS-ESI: m/z 376.2 observed (M + H)⁺ |
| 33 | 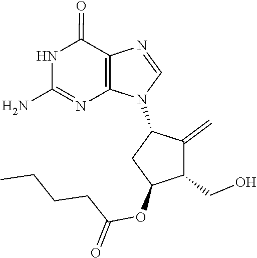 [SMILES: C=C1[C@H](CO)[C@@H](OC(=O)CCCC)C[C@@H]1n1cnc2c(=O)[nH]c(N)nc21] | ¹H NMR (400 MHz, DMSO-d₆) δ 10.59 (s, 1H), 7.71 (s, 1H), 6.43 (br s, 2H), 5.32-5.26 (m, 2H), 5.17 (s, 1H), 4.62 (s, 1H), 3.62 (d, J = 6.4 Hz, 2H), 2.72-2.64 (m, 1H), 2.55- 2.47 (m, 1H), 2.31 (t, J = 7.6 Hz, 2H), 2.23- 2.17 (m, 1H), 1.46-1.48 (m, 2H), 1.35-1.26 (m, 2H), 0.88 (t, J = 7.6 Hz, 3H). MS-ESI: m/z 362.1 observed (M + H)⁺ |
| 34 | 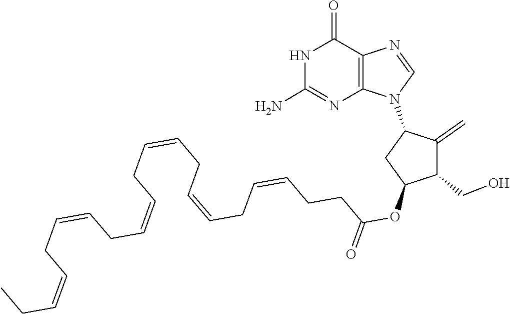 [SMILES: C=C1[C@H](CO)[C@@H](OC(=O)CC/C=C\C/C=C\C/C=C\C/C=C\C/C=C\C/C=C\CC)C[C@@H]1n1cnc2c(=O)[nH]c(N)nc21] | ¹H NMR (400 MHz, DMSO-d₆) δ 10.57 (s, 1H), 7.70 (s, 1H), 6.40 (br s, 2H), 5.38-5.26 (m, 14H), 5.16 (s, 1H), 5.01-4.94 (m, 1H), 4.61 (s, 1H), 3.65-3.58 (m, 2H), 2.83-2.76 (m, 10H), 2.72-2.64 (m, 1H), 2.54-2.50 (m, 1H), 2.49-2.22 (m, 4H), 2.15-2.06 (m, 1H), 2.03-1.99 (m, 2H), 0.91 (t, J = 7.6 Hz, 3H). MS-ESI: m/z 588.3 observed (M + H)⁺ |
| 35 | 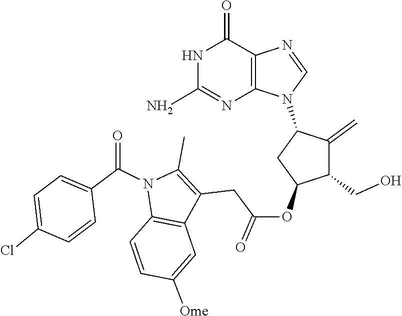 | ¹H NMR (400 MHz, DMSO-d₆) δ 10.52 (s, 1H), 7.69-7.61 (m, SH), 7.03 (d, J = 2.0 Hz, 1H), 6.92 (d, J = 7.2 Hz, 1H), 6.72 (d, J = 15.2 Hz, 1H), 6.38 (s, 2H), 5.36-5.25 (m, 2H), 5.20-5.17 (m, 1H), 5.01-4.99 (m, 1H), 4.65-4.59 (m, 1H), 3.80 (s, 2H), 3.76 (s, 3H), 3.69-3.55 (m, 2H), 2.76-2.71 (m, 1H), 2.58-2.50 (m, 1H), 2.30-2.17 (m, 4H). MS-ESI: m/z 617.2 observed (M + H)⁺ |
| 36 | 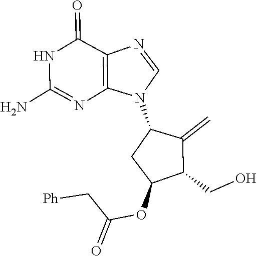 [SMILES: C=C1[C@H](CO)[C@@H](OC(=O)Cc2ccccc2)C[C@@H]1n1cnc2c(=O)[nH]c(N)nc21] | ¹H NMR (400 MHz, DMSO-d₆) δ 10.62 (s, 1H), 7.74 (s, 1H), 7.39-7.27 (m, 5H), 6.46 (br s, 2H), 5.37-5.30 (2H), 5.20 (s, 1H), 5.03 (t, J = 5.4 Hz, 1H), 4.65 (s, 1H), 3.72 (s, 2H), 3.64 (t, J = 6.0 Hz, 2H), 2.74-2.70 (m, 1H), 2.60-2.52 (m, 1H), 2.29-2.22 (m, 1H). MS-ESI: m/z 396.1 observed (M + H)⁺ |
| 37 | 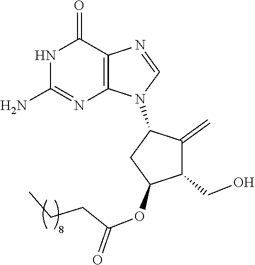 | ¹H NMR (400 MHz, DMSO-d₆) δ 10.59 (s, 1H), 7.71 (s, 1H), 6.43 (s, 2H), 5.59-5.57 (m, 2H), 5.31-5.26 (m, 2H), 5.16 (s, 1H), 4.99 (s, 1H), 4.61 (m, 1H), 3.62- 3.61 (m, 2H), 3.18 -3.14 (m, 1H), 2.70 (s, 1H), 2.31- 2.28 (m, 2H), 2.22-2.17 (m, 1H), 1.73-1.64 (m, 2H), 1.49-1.08 (m, 12H), 0.86-0.83 (m, 3H). MS-ESI: m/z 446.3 observed (M + H)⁺ |
| 38 | 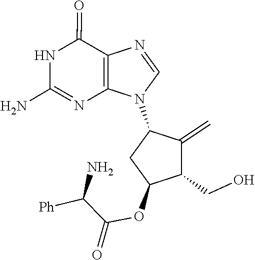 [SMILES: C=C1[C@H](CO)[C@@H](OC(=O)[C@H](N)c2ccccc2)C[C@@H]1n1cnc2c(=O)[nH]c(N)nc21] | |

### Pharmaceutical Composition

The disclosure also provides in various embodiments a pharmaceutical composition comprising a therapeutically effective amount of one or more compounds as described herein, or a pharmaceutically acceptable salt, stereoisomer, and/or tautomer thereof in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Table 1 or a pharmaceutically acceptable salt, stereoisomer, and/or tautomer thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated. the clinical condition of the subject, the cause of the disorder, the site of delivery of the agent, the method of administration, the scheduling of administration, and other factors known to medical practitioners.

The “therapeutically effective amount” of a compound or a pharmaceutically acceptable salt, stereoisomer, and/or tautomer thereof that is administered is governed by such considerations, and is the minimum amount necessary to inhibit reverse transcriptase activity, viral replication, production of viral proteins, or combinations thereof. Such amount may be below the amount that is toxic to normal cells or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg. Typical dose ranges are about 0.1 to about 400 mg/kg of patient body weight per day, with the typical initial range being about 50 to about 200 mg/kg/day. Oral unit dosage forms, such as tablets and capsules, may contain from about 0.1 mg to about 1000 mg of a compound (or a pharmaceutically acceptable salt, stereoisomer, or tautomer thereof) of the present disclosure. In another embodiment, such dosage forms contain from about 50 mg to about 500 mg of a compound (or a pharmaceutically acceptable salt, stereoisomer, or tautomer thereof) of the present disclosure. In yet another embodiment, such dosage forms contain from about 25 mg to about 200 mg of a compound (or a pharmaceutically acceptable salt, stereoisomer, or tautomer thereof) of the present disclosure. In still another embodiment, such dosage forms contain from about 10 mg to about 100 mg of a compound (or a pharmaceutically acceptable salt, stereoisomer, or tautomer thereof) of the present disclosure. In a further embodiment, such dosage forms contain from about 5 mg to about 50 mg of a compound (or a pharmaceutically acceptable salt, stereoisomer, or tautomer thereof) of the present disclosure. In any of the foregoing embodiments the dosage form can be administered one, two, three, or four times per day.

The compositions of the present disclosure can be administered orally, topically. parenterally, by inhalation or spray such as for pulmonary administration, or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

In another aspect, also encompassed are pharmaceutical compositions suitable for single unit dosages that comprise a compound of the disclosure or its pharmaceutically acceptable stereoisomer, salt, or tautomer and a pharmaceutically acceptable carrier.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of the compound.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, Jactose, calcium phosphate or sodium phosphate; granulating and disintegrating agents, for example, com starch, or alginic acid; binding agents, for example starch, gelatin or acacia, and lubricating agents, for example magnesium stearate, stearic acid or tale. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate. calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecacthyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil, or coconut oil, or in a mineral oil such as liquid paraffin, or in castor oil, or in combinations thereof. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

The compounds of the present disclosure may also be administered in the form of suppositories for rectal administration. These compositions can be prepared by mixing the compounds with a suitable non-irritating excipient which is solid at ordinary temperatures but liquid at the rectal temperature and will therefore melt in the rectum to release the compound. Such materials are cocoa butter and polyethylene glycols.

Compositions for parenteral administrations are administered in a sterile medium. Depending on the vehicle used and concentration the concentration of the compounds in the formulation, the parenteral formulation can either be a suspension or a solution containing dissolved compound. Adjuvants such as local anesthetics, preservatives and buffering agents can also be added to parenteral compositions.

In various embodiments, the present disclosure provides a formulation comprising a compound or pharmaceutically acceptable salt thereof as described herein. The compound is suspended in an aqueous solution comprising a water-soluble cellulose-based polymer and a non-ionic surfactant.

A water-soluble cellulose-based polymer, in accordance with various embodiments, is selected from the group consisting of alkyl celluloses, hydroxyalkyl celluloses, alkyl hydroxyalkyl celluloses, carboxyalkyl celluloses and salts thereof, and combinations thereof. Specific examples include, but are not limited to, methyl cellulose, ethyl cellulose, propyl cellulose, hydroxymethyl cellulose, hydroxyethyl cellulose, hydroxypropyl cellulose, hydroxypropylmethyl cellulose, carboxymethyl cellulose and salts thereof, and combinations thereof. In one embodiment, the water-soluble cellulose-based polymer is sodium carboxymethyl cellulose (CMC). The water-soluble cellulose-based polymer is present in an amount of about 0.1 to about 2.0% (w/v) or about 0.3 to about 0.8% (w/v). An exemplary amount is about 0.5% (w/v).

In additional embodiments, the non-ionic surfactant is selected from the group consisting of polyoxyethylene fatty acid esters, sorbitan fatty acid ester anhydrides, polyoxyethylene sorbitan esters, polyoxyethylene alkyl ethers, polyoxyethylene alkyl phenol ethers, polyoxyethylene fatty amines, polyoxyethylene alkyl amides, sorbitol anhydride fatty acid esters, and polyoxyethylene ethers. In some embodiments, the non-ionic surfactant is selected from polyoxyethylene sorbitan esters, such as polyoxyethylene (80) sorbitan monooleate (Tween-80). In various embodiments, the non-ionic surfactant is present in amount of about 0.1 to about 2.0% (w/v) or about 0.3 to about 0.8% (w/v). An exemplary amount is about 0.5% (w/v).

In an exemplary embodiment, the water-soluble cellulose-based polymer is CMC in an amount of about 0.5% (w/v) and the non-ionic surfactant is Tween-80 in an amount of about 0.5% (w/v).

### Methods of Use

The present disclosure further provides, in various embodiments, a method of inhibiting viral reverse transcriptase bioactivity. The method comprises contacting a virus expressing an enzyme with reverse transcriptase bioactivity with an effective amount or concentration of a compound as described herein.

The present disclosure further provides, in various embodiments, a method of prophylaxis of viremia or treatment of a viral infection in a patient wherein inhibition of a reverse transcriptase is medically indicated, comprising administering to the patient an effective amount or concentration of a compound as described herein. For example, the compound is administered in a formulation that provides for slow or controlled or sustained release of ETV from the compound. More specifically, the compound is formulated as aqueous suspensions or solutions as described herein, and it can be encapsulated in particles comprising, for example, poly(lactic-co-glycolic acid) (PLGA) for slow-release. In some embodiments, the viral infection is caused by HBV. The routes of administration for the compound include, but are not limited to, oral, parenteral and implants (drug delivery composition and device).

In an embodiment of the method for the treatment or prevention of HBV, the method further comprises at least one additional anti-HBV agent. The agent includes but is not limited to, adefovir dipivoxil, telbivudine, tenofovir disoproxil fumarate, tenofovir alafenamide fumarate, and lamivudine.

### EXAMPLES

### Abbreviations

The following abbreviations are used: tetrahydrofuran (THF), dichloromethane (DCM), Acetonitrile (MeCN), N,N-dimethylformamide (DMF), dimethylsulfoxide (DMSO), trifluoroacetic acid (TFA), tricthylamine (TEA). diisopropylethylamine (DIPEA), methanol (MeOH), Ethyl acetate (EtOAc), 4-Dimethylaminopyridine (DMAP), N-(3-Dimethylaminopropyl)-N′-cthylcarbodiimide hydrochloride (EDC●HCl), N,N′-Dicyclohexylcarbodiimide (DCC).

### General Examples for the Preparation of Compounds of the Present Disclosure

The starting materials and intermediates for the compounds described herein are prepared by the application or adaptation of the methods described below, their obvious chemical equivalents, or, for example, as described in literature such as The Science of Synthesis, Volumes 1-8. Editors E. M. Carreira et al. Thieme publishers (2001-2008). Details of reagent and reaction options are also available by structure and reaction searches using commercial computer search engines such as Scifinder (www.cas.org) or Reaxys (www.reaxys.com).

### Part I: Preparation of Intermediate-A

### Example 1: Synthesis of 2-amino-9-((1S,3R,4S)-3-(((tert-butyldimethylsilyl)oxy)methyl)-4-hydroxy-2-methylenecyclopentyl)-1H-purin-6(9H)-one (intermediate-A)

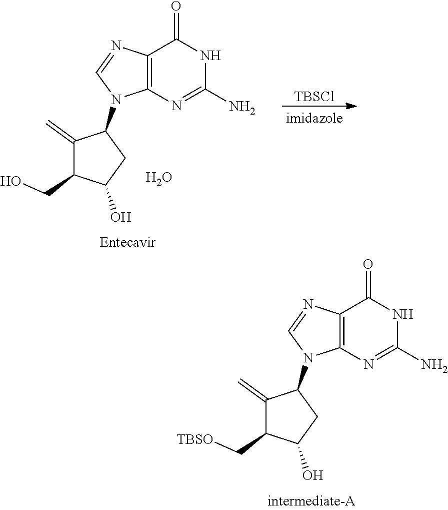

To a mixture of Entecavir hydrate (50.0 g, 170 mmol) and imidazole (34.6 g, 510 mmol) in N,N-dimethylformamid (1 L) was added teri-butyldimethylsilyl chloride (33.2 g, 220 mmol) at 0 to 5° C. After the addition the resulting mixture was stirred at 30° C. overnight. The mixture was poured into water (4 L) and filtered. The cake was washed with petroleum ether (400 mL) and then a solution of petroleum ether/ethyl acetate (9/1, 1000 mL). The obtained solid was dried in vacuum to give the desired intermediate −A (57.9 g, yield 87%) as white solids.

¹H NMR (400 MHZ, DMSO-d₆) δ 10.50 (s, 1H), 7.54 (s, 1H), 6.36 (br s, 2H), 5.28 (t, J=8.0 Hz, 1H), 5.07 (s, 1H), 4.86 (s, 1H), 4.53 (s, 1H), 4.11 (s, 1H), 3.64 (d, J=6.4 Hz, 2H), 2.48-2.43 (m, 1H), 2.13-2.07 (m, 1H), 2.00-1.94 (m, 1H), 0.81 (s, 9H), 0.08 (s, 6H).

### Part II: Preparation of Example Compounds

### Example 2: ((1R,3S,5S)-5-acetoxy-3-(2-amino-6-oxo-1,6-dihydro-9H-purin-9-yl)-2-methylenecyclopentyl)methyl acetate (1)

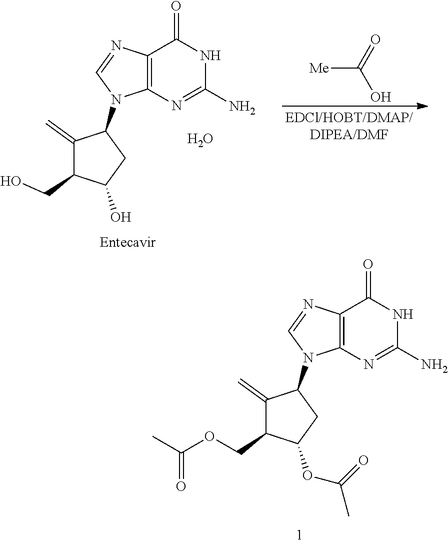

Prodrug 1 is prepared by using the procedure followed for the compound 2. LC-MS (ESI+): m/z 362.42 [M+H]+.

### Example 3: ((1R,2S,4S)-4-(2-amino-6-oxo-1H-purin-9(6H)-yl)-2-(isobutyryloxy)-3-methylenecyclopentyl)methyl isobutyrate (2)

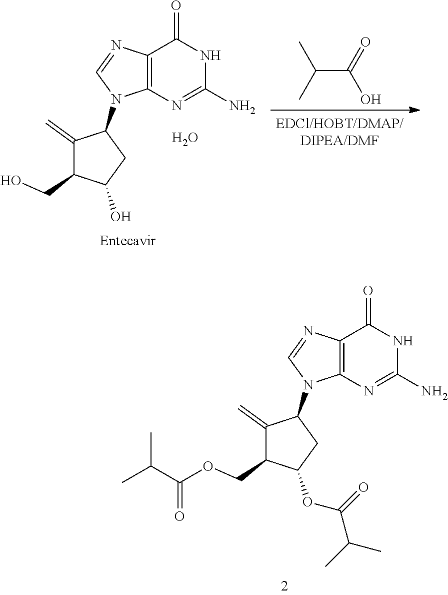

To a mixture of isobutyric acid (4.80 g, 54.2 mmol), 1-hydroxybenzotrizole (7.30 g, 54.2 mmol), 4-dimethylaminopyridine (200 mg, 1.36 mmol) and N,N-diisopropylethylamine (10.5 g, 81.4 mmol) in N,N-dimethylformamide (30 mL) was added N-(3-dimethylaminopropyl)-N′-ethylcarbodiimide hydrochloride (10.4 g, 54.2 mmol) and Entecavir hydrate (4.00 g, 13.6 mmol) at 0° C. The mixture was stirred at room temperature for 16 hours. Then the mixture was poured into water (50 mL) and extracted with ethyl acetate (50 mL) for 3 times. The combined organic phases were washed with 0.5 M hydrochloride aqueous solution (5 mL), saturated sodium bicarbonate aqueous solution (50 mL) and brine (50 mL), and then dried over sodium sulfate and filtered. The filtrate was concentrated in vacuum. The residue was triturated in methanol (5 mL) to give the desired 2 (4.1 g, yield 72%) as white solids.

LC-MS (ESI): R_{T}=3.042 min, mass calcd. 417.20 for C₂₀H₂₇N₅O₅, m/z found 418.2 [M+H]⁺.

### Example 4: (1S,3S,5R)-3-(2-amino-6-oxo-1H-purin-9(6H)-yl)-2-methylene-5-((pivaloyloxy)methyl)cyclopentyl pivalate (3)

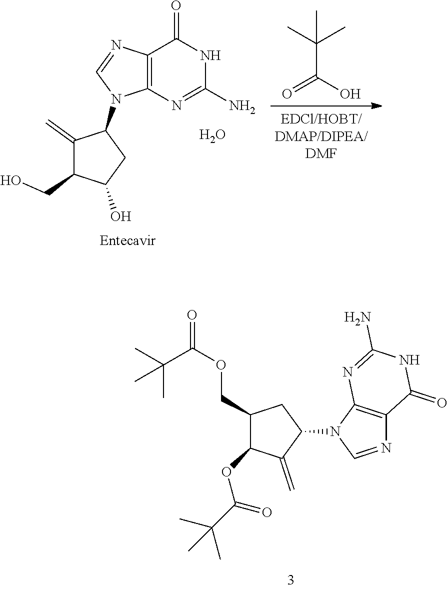

To a mixture of pivalic acid (5.54 g, 54.2 mmol), 1-hydroxybenzotrizole (7.32 g, 54.2 mmol), 4-dimethylaminopyridine (0.20 g, 1.36 mmol) and N,N-diisopropylethylamine (10.5 g, 81.4 mmol) in N,N-dimethylformamide (30 mL) was added N-(3-dimethylaminopropyl)-N′-ethylcarbodiimide hydrochloride (10.4 g, 54.2 mmol) and Entecavir hydrate (4.00 g, 13.6 mmol) at 0° C. The mixture was stirred at room temperature for 16 hours. Then the mixture was poured into water (50 mL) and extracted with ethyl acetate (50 mL) for 3 times. The combined organic phases were washed with 0.5 M hydrochloride aqueous solution (5 mL), saturated sodium bicarbonate aqueous solution (50 mL) and brine (50 mL), and then dried over sodium sulfate and filtered. The filtrate was concentrated in vacuum. The residue was triturated in methanol (20 mL) to give the desired 3 (5.16 g, yield 85%) as white solids.

LC-MS (ESI): R_{T}=2.358 min, mass calcd. 445.2 for C₂₂H₃₁N₅O₅, m/z found 446.2 [M+H]⁺.

### Example 5: ((1R,2S,4S)-4-(2-amino-6-oxo-1H-purin-9(6H)-yl)-2-((2-ethylbutanoyl)oxy)-3-methylenecyclopentyl)methyl 2-ethylbutanoate (4)

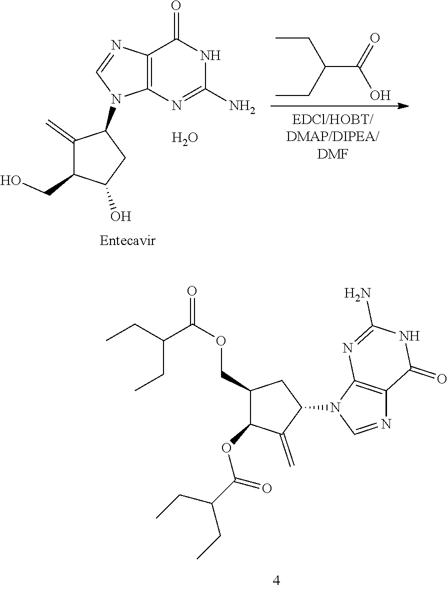

To a mixture of 2-ethylbutanoic acid (6.30 g, 54.2 mmol), 1-hydroxybenzotrizole (7.30 g, 54.2 mmol), 4-dimethylaminopyridine (200 mg, 1.36 mmol) and N,N-diisopropylethylamine (10.5 g, 81.4 mmol) in N,N-dimethylformamide (50 mL) was added N-(3-dimethylaminopropyl)-N′-ethylcarbodiimide hydrochloride (10.4 g, 54.2 mmol) and Entecavir hydrate (4.00 g, 13.6 mmol) at 0° C. The mixture was stirred at room temperature for 16 hours. Then the mixture was poured into water (150 mL) and extracted with ethyl acetate (150 mL) for 3 times. The combined organic phases were washed with 0.5 M hydrochloride aqueous solution (150 mL), saturated sodium bicarbonate aqueous solution (150 mL) and brine (150 mL), and then dried over sodium sulfate and filtered. The filtrate was concentrated in vacuum. The residue was triturated in methanol (30 mL) to give the desired 4 (4.30 g, yield 66%) as white solids.

LC-MS (ESI): R_{T}=3.606 min, mass calcd. 473.3 for C₂₄H₃₅N₅O₅, m/z found 474.2 [M+H]⁺.

### Example 6: ((1R,3S,5S)-3-(2-amino-6-oxo-1H-purin-9(6H)-yl)-2-methylene-5-(pentanoyloxy)cyclopentyl)methyl pentanoate (5) and ((1R,3S,5S)-3-(2-amino-6-oxo-1H-purin-9(6H)-yl)-5-hydroxy-2-methylenecyclopentyl)methyl pentanoate (26)

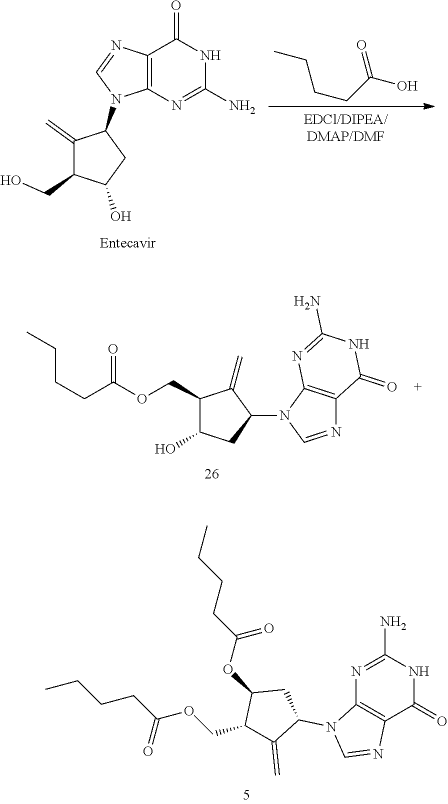

To a mixture of Entecavir hydrate (500 mg, 1.69 mmol), pentanoic acid (519 mg, 5.08 mmol), N-(3-dimethylaminopropyl)-N′-ethylcarbodiimide hydrochloride (1.30 g, 6.76 mmol) and 4-dimethylaminopyridine (21 mg, 0.17 mmol) in N,N-dimethylformamide (20 mL) was added N,N-diisopropylethylamine (874 mg, 6.76 mmol) at 0° C. The mixture was stirred at room temperature for 16 hours. The reaction mixture was poured into water (150 mL). EtOAc (100 mL×2) was added to extract the desired compound. The combined organic layers were washed with saturated NH₄Cl aqueous solution (150 mL) and brine (200 mL) twice, dried over Na₂SO₄ and filtered. The filtrate was concentrated to give a residue. The residue was purified by column C18 (Mobile Phase A: water, Mobile Phase B: acetonitrile, Gradient: 5-60% (% B)) to give the desired 26 (40 mg, yield 6.5%) as white solid and 5 (190 mg, yield 25%) as white solid.

26: LC-MS (ESI): R_{T}=2.941 min, mass calcd. 361.4 for C₁₇H₂₃N₅O₄, m/z found 362.2 [M+H]⁺.

5: LC-MS (ESI): R_{T}=3.836 min, mass calcd. 445.5 for C₂₂H₃₁N₅O₅, m/z found 446.2 [M+H

### Example 7A: (1S,2R,4R)-4-(2-amino-6-oxo-1H-purin-9(6H)-yl)-2-(((tert-butyldimethylsilyl)oxy)methyl)-3-methylenecyclopentyl pentanoate (intermediate-B)

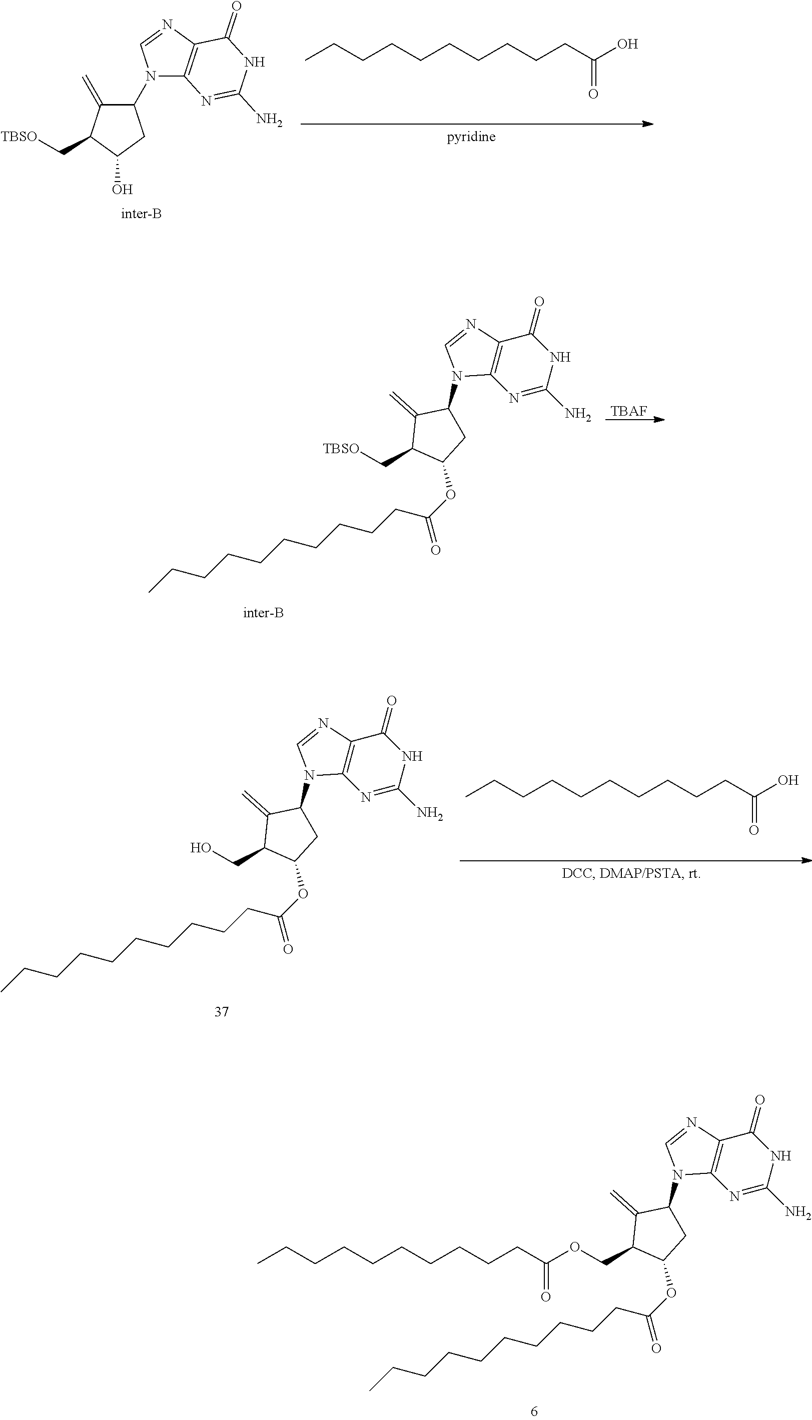

To a mixture of 2-amino-9-((1S,3R,4S)-3-(((tert-butyldimethylsilyl)oxy)methyl)-4-hydroxy-2-methylenecyclopentyl)-1H-purin-6(9H)-one (intermediate-A) (1.20 g. 3.06 mmol), 4-dimethylaminopyridine (200 mg, cat.) and p-toluenesulfonic acid (200 mg, cat.) in N,N-dimethylformamide (20 mL) was added undecanoic acid (14 g, 6.12 mmol) at 25° C. After stirred at room temperature for 48 hours the mixture was poured into water (50 mL) and extracted with ethyl acetate (100 mL) for 3 times. The combined organic phases were washed with brine (50 mL), and then dried over sodium sulfate and filtered. The filtrate was concentrated in vacuum. The residue was purified by column chromatography on silica gel (dichloromethane:methanol=20:1 to 10:1) to give the desired compound intermediate-B (185 mg, vield 76%) as white solids.

### Example 7B: (1S,2R,4S)-4-(2-amino-6-oxo-1H-purin-9(6H)-yl)-2-(hydroxymethyl)-3-methylenecyclopentyl undecanoate (37)

To a solution of (1S,2R,4R)-4-(2-amino-6-oxo-1H-purin-9(6H)-yl)-2-(((tert-butyldimethylsilyl)oxy)methyl)-3-methylenecyclopentyl pentanoate (intermediate-B) (1.40 g, 2.50 mmol) in tetrahydrofuran (20 mL) was added 1 M TABF in tetrahydrofuran (5 mL, 5 mmol) at 0° C. The mixture was stirred at room temperature for 3 hours. The mixture was concentrated and the residue was poured into water (50 mL) and extracted with ethyl acetate (50 mL) for 3 times. The combined organic phases were washed with brine (50 mL), and then dried over sodium sulfate and filtered. The filtrate was concentrated in vacuum. The residue was purified by column chromatography on silica gel (dichloromethane:methanol=30:1 to 10:1) to give the desired compound 37 (1.1 g, vield 99%) as white solids.

¹H NMR (400 MHz, DMSO-d₆) δ 10.59 (s, 1H), 7.71 (s, 1H), 6.43 (s, 2H), 5.59-5.57 (m, 2H), 5.31-5.26 (m, 2H), 5.16 (s, 1H), 4.99 (s, 1H), 4.61 (m, 1H), 3.62-3.61 (m, 2H), 3.18 -3.14 (m, 1H), 2.70 (s, 1H), 2.31-2.28 (m, 2H), 2.22-2.17 (m, 1H), 1.73-1.64 (m, 2H), 1.49-1.08 (m, 12H), 0.86-0.83 (m, 3H).

### Example 7C: ((1R,3S,5S)-3-(2-amino-6-oxo-1H-purin-9(6H)-yl)-2-methylene-5-(undecanoyloxy)cyclopentyl)methyl undecanoate (6)

To a solution of (1S,2R,4S)-4-(2-amino-6-oxo-1H-purin-9(6H)-yl)-2-(hydroxymethyl)-3-methylenecyclopentyl undecanoate (37) (400 mg, 0.900 mmol). DMAP/p-toluenesulfonic acid (20 mg/20 mg) in DMF (5 mL) was added DCC (556 mg, 2.70 mmol) at room temperature for 48 hours. Then the mixture was poured into water (50 mL) and extracted with EtOAc (20 mL) for three times. The combined organic phases were washed with water (30 mL) twice and brine (30 mL), dried over Na₂SO₄ and concentrated. The residue was purified by silica gel chromatography using DCM:MeOH=20:1 to 10:1 as eluent to give the title compound 6 (180 mg, yield 33%) as white solids.

LC-MS (ESI): R_{T}=3.739 min, mass calcd. 613.4 for C₃₄H₅₅N₅O₅, m/z found 614.5 [M+H]⁺.

### Example 8A: (9Z,12Z)-(1S,2R,4S)-4-(2-amino-6-oxo-1H-purin-9(6H)-yl)-2-(((tert-butyldimethylsilyl)oxy)methyl)-3-methylenecyclopentyl octadeca-9,12-dienoate (inter-C)

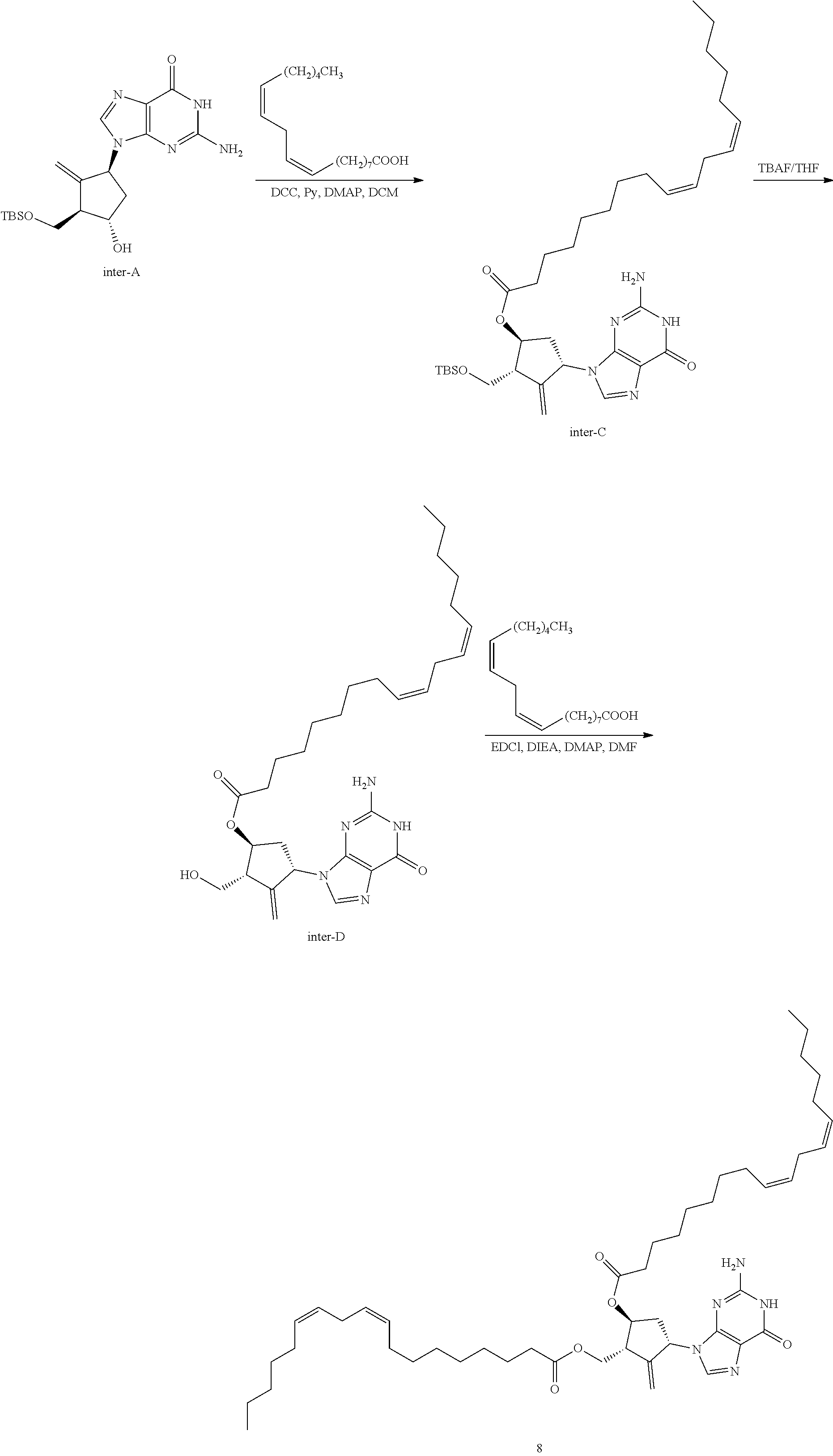

To a mixture of 2-amino-9-((1S,3R,4S)-3-(((tert-butyldimethylsilyl)oxy)methyl)-4-hydroxy-2-methylenecyclopentyl)-1H-purin-6(9H)-one (inter-A) (1.50 g, 3.83 mmol), (9Z,12Z)-octadeca-9,12-dienoic acid (3.23 g, 11.5 mmol), pyridine (3.03 g, 38.3 mmol) and 4-dimethylaminopyridine (46 mg, 0.38 mmol) in dichloromethane (300 mL) was added N,N′-dicyclohexylcarbodiimide (2.37 g, 11.5 mmol) at 25° C. After stirred at room temperature for 16 hours the mixture was concentrated in vacuum. The residue was purified by column chromatography on silica gel (dichloromethane:methanol=20:1 to 10:1) to give the desired compound inter-C (2.70 g, 89% purity, yield 96%) as white solids.

LC-MS (ESI): R_{T}=4.030 min, mass calcd. 653.4 for C₃₆H₅₉N₅O₄Si, m/z found 654.3 [M+H]⁺.

### Example 8B: (9Z,12Z)-(1S,2R,4S)-4-(2-amino-6-oxo-1H-purin-9(6H)-yl)-2-(hydroxymethyl)-3-methylenecyclopentyl octadeca-9,12-dienoate (inter-D)

To a solution of (9Z,12Z)-(1S,2R,4S)-4-(2-amino-6-oxo-1H-purin-9(6H)-yl)-2-(((tert-butyldimethylsilyl)oxy)methyl)-3-methylenecyclopentyl octadeca-9,12-dienoate (2) (2.70 g, 3.6 mmol) in tetrahydrofuran (30 mL) was added 1 M TABF in tetrahydrofuran (25 mL, 25 mmol) at room temperature. The mixture was stirred at room temperature for 16 bours. The mixture was concentrated in vacuum to give a residue. The residue was purified by column chromatography on silica gel (dichloromethane:methanol=15:1) to give the desired compound inter-D (580 mg, yield 30%) as gray solids.

¹H NMR (300 MHz, DMSO-d₆) δ 10.66 (s, 1H), 7.74 (s, 1H), 6.49 (br s, 2H), 5.41-5.30 (m, 7H), 5.19 (s, 1H), 5.04 (t, J=5.4 Hz, 1H), 4.65 (s, 1H), 3.65 (t, J=5.7 Hz, 2H), 2.77-2.73 (m, 3H), 2.59-2.49 (m. 1H), 2.32 (t, J =7.2 Hz, 2H), 2.26-2.19 (m, 1H), 2.05-2.00 (m, 4H), 1.63-1.53 (m, 2H), 1.35-1.29 (m, 14H), 0.88 (t, J=6.9 Hz, 3H).

### Example 8C: (9Z,12Z)-((1R,3S,5S)-3-(2-amino-6-oxo-1H-purin-9(6H)-yl)-2-methylene-5-((9Z,12Z)-octadeca-9,12-dienoyloxy)cyclopentyl)methyl octadeca-9,12-dienoate (8)

To a solution of (9Z, 12Z)-octadeca-9.12-dienoic acid (445 mg, 1.58 mmol), N-(3-dimethylaminopropyl)-N′-ethylcarbodiimide hydrochloride (403 mg, 2.10 mmol) and 4-dimethylaminopyridine (26 mg, 0.21 mmol) in N,N-dimethylformamide (50 mL) at 0° C. was added N,N-diisopropylethylamine (407 mg, 3.15 mmol). The resulting mixture was stirred at 0° C. for 30 minutes. A solution of (9Z,12Z)-(1S,2R,4S)-4-(2-amino-6-oxo-1H-purin-9(6H)-yl)-2-(hydroxymethyl)-3-methylenecyclopentyl octadeca-9,12-dienoate (570 mg, 1.05 mmol) was added. The mixture was stirred at room temperature ovemight. Then the mixture was concentrated in vacuum. The residue was purified by column chromatography on silica gel (dichloromethane:methanol=10:1) and pre-TLC (dichloromethane:methanol=10:1) to give the desired 8 (250 mg, yield 30%) as gray semi-solids.

LC-MS (ESI): R_{T}=7.179 min, mass calcd. 801.6 for C₄₈H₇₅N₅O₅, m/z found 802.6 [M+H]⁺.

### Example 9: (4Z,7Z,10Z,13Z,16Z,19Z)-(1S,2R,4S)-4-(2-amino-6-oxo-1H-purin-9(6H)-yl)-2-(((4Z,7Z,10Z,13Z,16Z,19Z)-docosa-4,7,10,13,16,19-hexaenoyloxy)methyl)-3-methylenecyclopentyl docosa-4,7,10,13,16,19-hexaenoate (9)

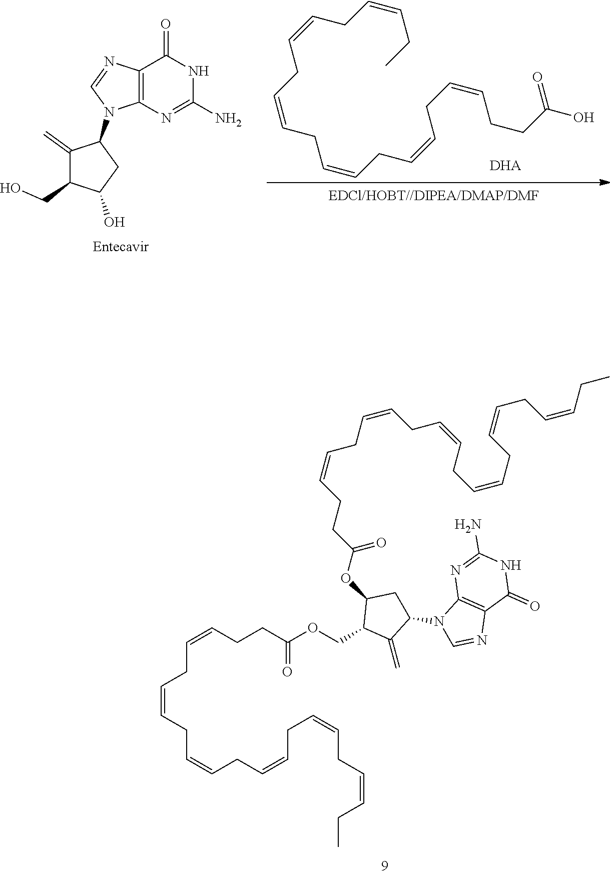 [SMILES: C=C1[C@H](CO)[C@@H](O)C[C@@H]1n1cnc2c(=O)[nH]c(N)nc21 ; CC/C=C\C/C=C\C/C=C\C/C=C\C/C=C\C/C=C\CCC(=O)O ; "DHA" ; C=C1[C@H](COC(=O)CC/C=C\C/C=C\C/C=C\C/C=C\C/C=C\C/C=C\CC)[C@@H](OC(=O)CC/C=C\C/C=C\C/C=C\C/C=C\C/C=C\C/C=C\CC)C[C@@H]1n1cnc2c(=O)[nH]c(N)nc21]

A mixture of DHA (44.5 g, 136 mmol), 1-hydroxybenzotrizole (27.5 g, 203 mmol), 4-dimethylaminopyridine (415 mg, 3.40 mmol) and N,N-diisopropylethylamine (43.8 g, 339 mmol) in N,N-dimethylformamide (300 mL) was stirred at 0° C. for 45 minutes. N-(3-dimethylaminopropyl)-N′-ethylcarbodiimide hydrochloride (39.0 g, 203 mmol) was added. After 15 minutes, entecavir hydrate (10.0 g, 33.9 mmol) was added at 0° C. The mixture was stirred at room temperature for 2 days. Then the mixture was poured into water (1000 mL) and extracted with ethyl acetate (500 mL) for 3 times. The combined organic phases were washed with 0.5 M hydrochloride aqueous solution (500 mL), saturated sodium bicarbonate aqueous solution (500 mL) and brine (500 mL), dried over sodium sulfate and filtered. The filtrate was concentrated in vacuum. The residue was purified by column C18 (Mobile Phase A: water, Mobile Phase B: acetonitrile, Gradient: 5-100% (% B) and then 100% methanol) to give the desired 9 (23.6 g, yield 77%) as brown semi-solids.

LC-MS (ESI): R_{T}=11.775 min, mass calcd. 897.6 for C₅₆H₇₅N₅O₅, m/z found 898.6 [M+H]⁺

### Example 10: (1S,2R,4S)-4-(2-amino-6-oxo-1H-purin-9(6H)-yl)-2-((2-(1-(4-chlorobenzoyl)-5-methoxy-2-methyl-1H-indol-3-yl)acetoxy)methyl)-3-methylenecyclopentyl 2-(1-(4-chlorobenzoyl)-5-methoxy-2-methyl-1H-indol-3-yl)acetate (10)

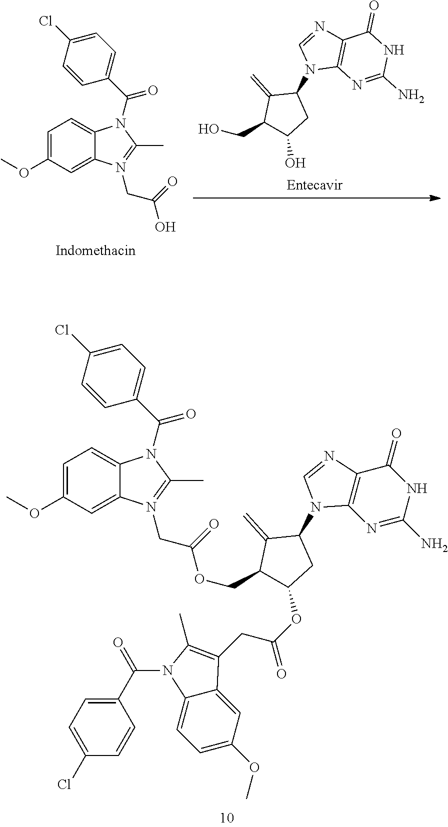

To a mixture of Indomethacin (35.7 g, 100 mmol), Entecavir hydrate (10.0 g, 33.9 mmol), 4-dimethylaminopyridine (366 mg, 3 mmol) and N,N-diisopropylethylamine (51.6 g, 400 mmol) in N,N-dimethylformamide (200 mL) was added N-(3-dimethylaminopropyl)-N′-ethylcarbodiimide hydrochloride (38 g, 200 mmol) and HOBT (27.0 g, 0.200 mol). The mixture was stirred at room temperature for 18 hours. Then the mixture was poured into water (600 mL) and extracted with ethyl acetate (200 mL) for 3 times. The combined organic phases were washed with 1 M hydrochloride aqueous solution (200 mL) for 3 times, saturated sodium bicarbonate aqueous solution (200 mL) and brine (100 mL), and then dried over sodium sulfate and filtered. The filtrate was concentrated in vacuum. The residue was purified by column C18 (Mobile Phase A: water, Mobile Phase B: acetonitrile, Gradient: 5-100% (% B)) to give the desired 10 (14.0 g, yield 44%) as yellow solids.

LC-MS (ESI): R_{T}=3.632 min, mass calcd. 955.3 for C₅₀H₄₃Cl₂N₇O₉, m/z found 956.2 [M+H]⁺.

### Example 11: ((1R,3S,5S)-3-(2-amino-6-oxo-1H-purin-9(6H)-yl)-2-methylene-5-(2-phenylacetoxy)cyclopentyl)methyl 2-phenylacetate (11)

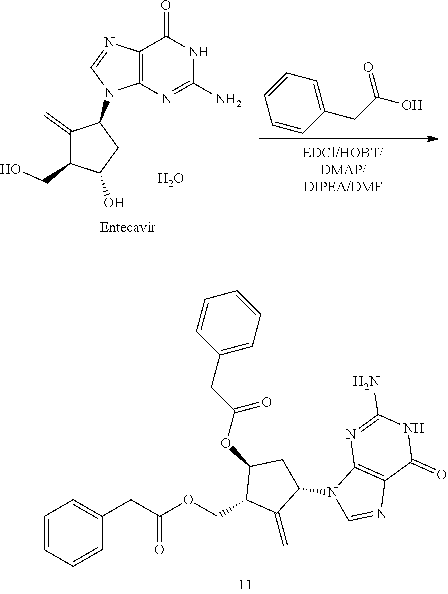

To a mixture of 2-phenylacetic acid (14.8 g, 108 mmol), 1-hydroxybenzotrizole (22.0 g, 163 mmol), 4-dimethylaminopyridine (330 mg, 2.7 mmol) and N,N-diisopropylethylamine (35.1 g, 271 mmol) in N,N-dimethylformamide (50 mL) was added N-(3-dimethylaminopropyl)-N′-ethylcarbodiimide hydrochloride (31.19 g, 163 mmol) and Entecavir hydrate (8.00 g, 27.1 mmol) at 0° C. The mixture was stirred at room temperature for 16 hours. Then the mixture was poured into water (600 mL) and extracted with ethyl acetate (300 mL) for 3 times. The combined organic phases were washed with 0.5 M hydrochloride aqueous solution (300 mL), saturated sodium bicarbonate aqueous solution (300 mL) and brine (300 mL), and then dried over sodium sulfate and filtered. The filtrate was concentrated in vacuum. The residue was triturated in methanol (30 mL) to give the desired 11 (11.3 g, yield 84%) as white solids.

LC-MS (ESI): R^{T}=3.554 min, mass calcd. 513.2 for C₂₈H₂₇N₅O₅, m/z found 514.2 [M+H]⁺.

### Example 12A: (R)-(1S,2R,4S)-4-(2-amino-6-oxo-1H-purin-9(6H)-yl)-2-(((R)-2-((tert-butoxycarbonyl)amino)-2-phenylacetoxy)methyl)-3-methylenecyclopentyl 2-((tert-butoxycarbonyl)amino)-2-phenylacetate (inter-E)

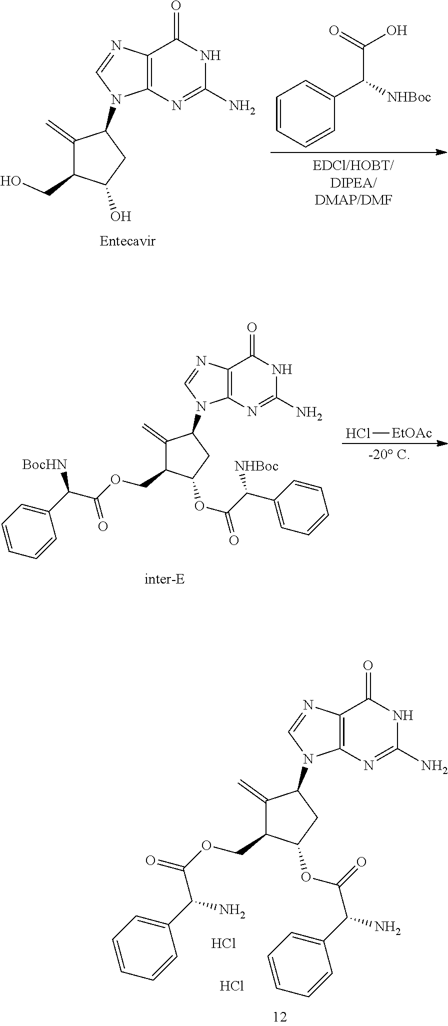

To a mixture of Entecavir hydrate (10.0 g, 33.9 mmol), (R)-2-((tert-butoxycarbonyl)amino)-2-phenylacetic acid (30.0 g, 119 mmol) 4-dimethylaminopyridine (1.00 g, 8.20 mmol) and N,N-diisopropylethylamine (40 g, 310 mmol) in N,N-dimethylformamide (200 mL) was added N-(3-dimethylaminopropyl)-N′-ethylcarbodiimide hydrochloride (40.0 g, 209 mmol) and HOBT (22.0 g, 162 mmol). The mixture was stirred at room temperature for 16 hours. Then the mixture was poured into water (800 mL) and extracted with ethyl acetate (250 mL) for 3 times. The combined organic phases were washed with 1 M hydrochloride aqueous solution (200 mL) for 3 times, saturated sodium bicarbonate aqueous solution (100 mL) and brine (100 mL), and then dried over sodium sulfate and filtered. The filtrate was concentrated in vacuum to give the desired 2 (28.0 g, yield 100%) as white solids.

### Example 12B: (R)-((1R,3S,5S)-5-((R)-2-amino-2-phenylacetoxy)-3-(2-amino-6-oxo-1H-purin-9(6H)-yl)-2-methylenecyclopentyl)methyl 2-amino-2-phenylacetate dihydrochloride (12)

A solution of (R)-(1S,2R,4S)-4-(2-amino-6-oxo-1H-purin-9(6H)-yl)-2-(((R)-2-((tert-butoxycarbonyl)amino)-2-phenylacetoxy)methyl)-3-methylenecyclopentyl 2-((tert-butoxycarbonyl)amino)-2-phenylacetate (inter-E) (28.0 g, 37.7 mmol) in 4 M hydrochloride in ethyl acetate (250 mL) was stirred at −20° C.for 2 hours. The mixture was filtered and the cake was washed with ethyl acetate (100 mL) to give the desired 12 (20.0 g, yield 86%) as white solids.

LC-MS (ESI): R_{T}=3.020 min, mass calcd. 543.2 for C₂₈H₂₉N₇O₅, m/z found 544.2 [M+H]⁺.

### Example 13A: (1S,2R,4S)-4-(2-amino-6-oxo-1H-purin-9(6H)-yl)-2-(((tert-butyldimethylsilyl)oxy)methyl)-3-methylenecyclopentyl 3-methylbutanoate (inter-F)

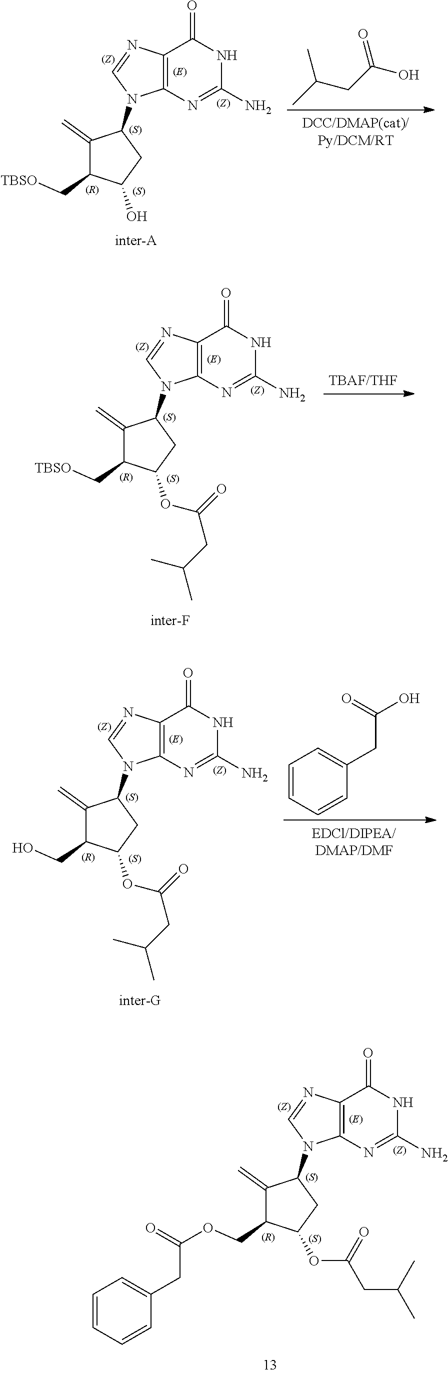

To a mixture of 2-amino-9-((1S,3R,4S)-3-(((tert-butyldimethylsilyl)oxy)methyl)-4-hydroxy-2-methylenecyclopentyl)-1H-purin-6(9H)-one inter-A (2.0 g, 5.1 mmol), 3-methylbutanoic acid (1.04 g, 10.2 mmol), pyridine (1.62 g, 20.4 mmol) and 4-dimethylaminopyridine (62 mg, 0.51 mmol) in dichloromethane (250 mL) was added N,N′-dicyclohexylcarbodiimide (2.11 g, 10.2 mmol) at 0° C. After stirred at room temperature for 16 hours the mixture was poured into water (100 mL). The mixture was extracted with ethyl acetate (100 mL) twice. The combined organic phases were washed with brine (100 mL), and then dried over sodium sulfate and filtered. The filtrate was concentrated under vacuum. The residue was purified by column chromatography on silica gel (dichloromethane:methanol=20:1) to give the desired compound inter-F (2.18 g, yield 91%) as white solids.

¹H NMR (300 MHz, DMSO-d₆) δ 10.64 (s, 1H), 7.69 (s, 1H), 6.49 (br s, 2H), 5.36-5.24 (m, 3H), 4.68 (s, 1H), 3.83-3.77 (m, 2H), 2.80-2.75 (m, 1H), 2.56-2.46 (m, 1H), 2.27-2.21 (m, 3H), 2.10-1.97 (m), 1H), 0.95 (s, 3H), 0.93 (s, 3H), 0.91 (s, 9H), 0.11 (s, 6H).

### Example 13B: (1S,2R,4S)-4-(2-amino-6-oxo-1H-purin-9(6H)-yl)-2-(hydroxymethyl)-3-methylenecyclopentyl 3-methylbutanoate (inter-G)

To a solution of (1S,2R,4S)-4-(2-amino-6-oxo-1H-purin-9(6H)-yl)-2-(((tert-butyldimethylsilyl)oxy)methyl)-3-methylenccyclopentyl 3-methylbutanoate (inter-F) (2.10 g. 4.40 mmol) in tetrahydrofuran (80 mL) was added 1M TABF in tetrahydrofuran (15 mL, 15 mmol) at room temperature. The mixture was stirred at room temperature for 16 hours. The mixture was poured into water (100 mL). The mixture was extracted with ethyl acetate (100 mL) twice. The combined organic phases were washed with brine (100 mL), and then dried over sodium sulfate and filtered. The filtrate was concentrated under vacuum. The residue was purified by column chromatography on silica gel (dichloromethane:methanol=20:1) to give the desired compound inter-G (780 mg, yield 48%) as white solids.

LC-MS (ESI): R_{T}=1.955 min, mass calcd. 361.4 for C₁₇H₂₃N₅O₄, m/z found 362.1 [M+H]⁺.

### Example 13C: (1S,2R,4S)-4-(2-amino-6-oxo-1H-purin-9(6H)-yl)-3-methylene-2-((2-phenylacetoxy)methyl)cyclopentyl 3-methylbutanoate (13)

To a mixture of (1S,2R,4S)-4-(2-amino-6-oxo-1H-purin-9(6H)-yl)-2-(hydroxymethyl)-3-methylenecyclopentyl 3-methylbutanoate (inter-G) (300 mg, 0.8311 mmol), N-(3-dimethylaminopropyl)-N′-ethylcarbodiimide hydrochloride (527 mg, 2.75 mmol), 4-dimethylaminopyridine (10 mg. 0.08 mmol) and 2-phenylacetic acid (278 mg, 2.04 mmol) in N,N-dimethylformamide (20 mL) was added N,N-diisopropylethylamine (350 mg. 2.71 mmol) at 0° C. The mixture was stirred at room temperature overnight. Then the mixture was poured into water (100 mL) and extracted with ethyl acetate (50 mL) for 3 times. The combined organic phases were dried over sodium sulfate and filtered. The filtrate was concentrated in vacuum. The residue was purified by column (dicholormehane:methanol=20:1) to give the desired 13 (205 mg, yield 52%) as white solids.

LC-MS (ESI): R_{T}=3.640 min, mass calcd. 479.2 for C₂₅H₂₉N₅O₅, m/z found 580.2 [M+H]+.

### Examples 14-17

Compounds 14-17 were prepared by using the procedure followed for the compound 13.

### Example 18A: ((1R,2S,4S)-4-(2-amino-6-oxo-1H-purin-9(6H)-yl)-2-hydroxy-3-methylenecyclopentyl)methyl 2-(1-(4-chlorobenzoyl)-5-methoxy-2-methyl-1H-indol-3-yl)acetate (28)

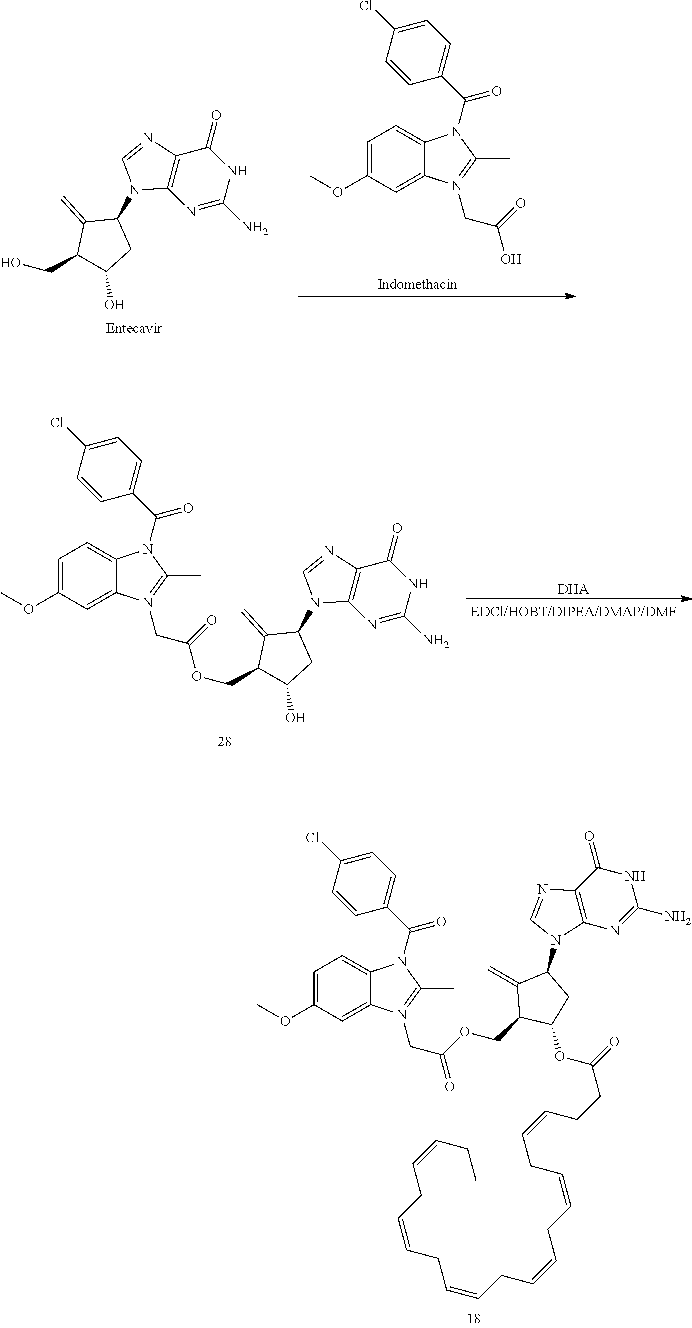

To a mixture of Indomethacin (60.5 g, 169 mmol), 1-hydroxybenzotrizole (45.8 g, 340 mmol), 4-dimethylaminopyridine (2.07 g, 20 mmol) and N,N-diisopropylethylamine (102.8 g, 800 mmol) in N,N-dimethylformamide (500 mL) was added N-(3-dimethylaminopropyl)-N′-ethylcarbodiimide hydrochloride (64.98 g, 340 mmol) and Entecavir hydrate (50.0 g, 169 mmol) at 0° C. The mixture was stirred at room temperature for 16 hours. Then the mixture was poured into water (1 L) and extracted with ethyl acetate (1 L) for 3 times. The combined organic phases were washed with 0.5 M hydrochloride aqueous solution (500 mL), saturated sodium bicarbonate aqueous solution (500 mL) and brine (500 mL), and then dried over sodium sulfate and filtered. The filtrate was concentrated in vacuum. The residue was triturated in methanol (50 mL) to give the desired 28 (35 g, yield 33%) as white solids.

LC-MS (ESI): R_{T}=3.957 min, mass calcd. 616.2 for C₃₁H₂₉ClN₆O₆, m/z found 617.1 [M+H]⁺.

### Example 18B: (4Z,7Z,10Z,13Z,16Z,19Z)-(1S,2R,4S)-4-(2-amino-6-oxo-1H-purin-9(6H)-yl)-2-((2-(1-(4-chlorobenzoyl)-5-methoxy-2-methyl-1H-indol-3-yl)acetoxy)methyl)-3-methylenecyclopentyl docosa-4,7,10,13,16,19-hexaenoate (18)

A mixture of DHA (23.4 g, 71.3 mmol). I-hydroxybenzotrizole (12.0 g, 89.0 mmol), 4-dimethylaminopyridine (217 mg, 1.78 mmol) and N,N-diisopropylethylamine (16.1 g, 125 mmol) in N,N-dimethylformamide (300 mL) was stirred at 5° C. for 30 minutes. N-(3-dimethylaminopropyl)-N′-ethylcarbodiimide hydrochloride (17.1 g, 89.0 mmol) was added. After 10 minutes, ((1R,3S,5S)-3-(2-amino-6-oxo-1H-purin-9(6H)-yl)-5-hydroxy-2-methylenecyclopentyl)methyl 2-(1-(4-chlorobenzoyl)-5-methoxy-2-methyl-1H-indol-3-yl)acetate (28) (11.0 g, 17.8 mmol) was added at 0° C. The mixture was stirred at room temperature overnight Another mixture of DHA (23.4 g, 71.3 mmol). 1-hydroxybenzotrizole (12.0 g, 89.0 mmol), 4-dimethylaminopyridine (217 mg, 1.78 mmol) and N,N-diisopropylethylamine (16.09 g, 124.6 mmol) in N,N-dimethylformamide (300 mL) was stirred at 5° C. for 30 minutes. N-(3-dimethylaminopropyl)-N′-ethylcarbodiimide hydrochloride (17.1 g, 89.0 mmol) was added. After 30 minutes, the new mixtue was added into the previous mixture. Stirring was continued at room temperature for 2 days. Then the mixture was poured into water (1000 mL) and extracted with ethyl acetate (500 mL) for 3 times. The combined organic phases were washed with 1 M hydrochloride aqueous solution (500 mL) twice and brine (500 mL), and then dried over sodium sulfate and filtered. The filtrate was concentrated in vacuum. The residue was purified by column chromatography on silica gel (dichloromethane:methanol=20:1 to 10:1) to give the desired 18 (7.60 g, yield 46%) as yellow solids.

LC-MS (ESI): R_{T}=3.344 min, mass calcd. 926.4 for C₅₃H₅₉ClN₆O₇, m/z found 927.4 [M+H]⁺.

### Example 19

Compound 19 was prepared by using the procedure followed for the compound 18 employing (R)-2-((tert-butoxycarbonyl)amino)-2-phenylacetic acid followed by deprotection using HCl-EtOAc at 0° C.

### Example 20A: (1S,2R,4S)-4-(2-amino-6-oxo-1H-purin-9(6H)-yl)-2-(((tert-butyldimethylsilyl)oxy)methyl)-3-methylenecyclopentyl hexanoate (inter-H)

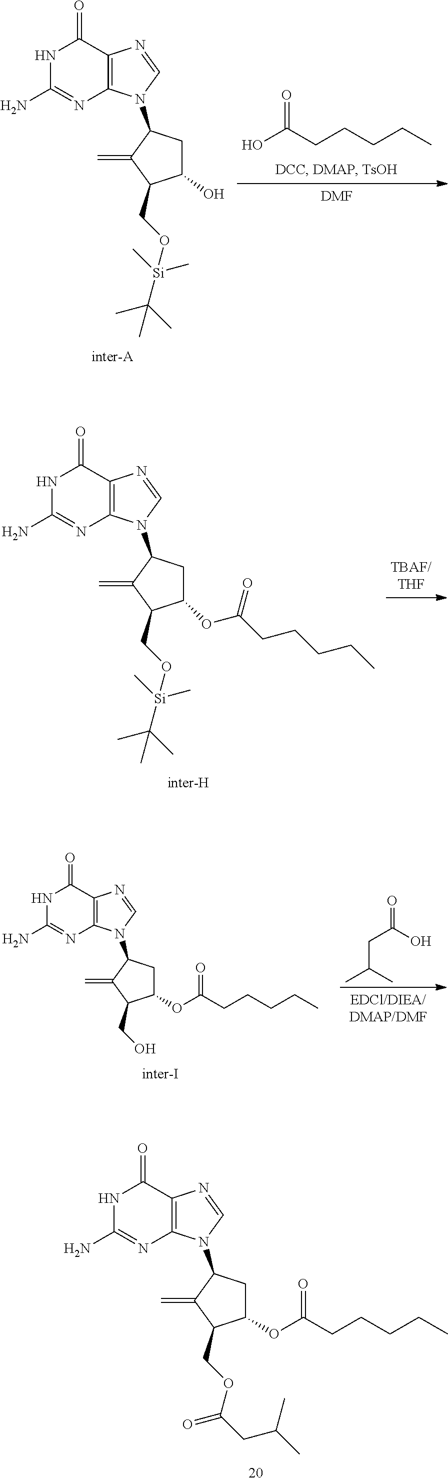

To a solution of 2-amino-9-((1S,3R,4S)-3-(((tert-butyldimethylsilyl)oxy)methyl)-4-hydroxy-2-methylenecyclopentyl)-1H-purin-6(9H)-one (inter-A) (1.50 g, 3.84 mmol) in DMF (40 mL) was added DMAP (20 mg) and TsOH (10 mg). Hexanoic acid (445 mg, 3.84 mmol) and DCC (790 mg 3.84 mmol) was added. The mixture was stirred at room temperature for 16 hours. The 2nd batch of Hexanoic acid (445 mg, 3.84 mmol) and DCC (790 mg 3.84 mmol) was added. The mixture was stirred at room temperature for 16 hours. The 3rd batch of Hexanoic acid (445 mg, 3.84 mmol) and DCC (790 mg 3.84 mmol) was added. The mixture was stirred at room temperature for 16 hours. The 4th batch of Hexanoic acid (445 mg, 3.84 mmol) and DCC (790 mg 3.84 mmol) was added. The mixture was stirred at room temperature for 72 hours. Then the mixture was poured into water (100 mL) and extracted with ethyl acetate (30 mL) for 3 times. The combined organic phases were washed with water (10 mL) and brine (10 mL), and then dried over sodium sulfate and filtered. The filtrate was concentrated in vacuum. The residue was purified by chromatography on silica gel (dichloromethane:methanol=50:1 to 20:1 to 10:1) to give the desired compound inter-H (1.65 g, vield 88%) as white solid.

¹H NMR (400 MHZ, DMSO-d₆) δ 10.59 (s, 1H), 7.65 (s, 1H), 6.44 (br s, 2H), 5.32-5.21 (m, 3H), 5.03-4.94 (m, 1H), 4.65-4.64 (m, 1H), 3.80-3.78 (m, 2H), 2.72-2.69 (m, 1H), 2.50-2.43 (m, 1H), 2.32-2.28 (m, 2H), 2.24-2.19 (m, 1H), 1.55-1.49 (m, 2H), 1.29-1.24 (m, 4H), 0.90-0.84 (m, 12H), 0.09-0.08 (m, 6H).

### Example 20B: (1S,2R,4S)-4-(2-amino-6-oxo-1H-purin-9(6H)-yl)-2-(hydroxymethyl)-3-methylenecyclopentyl hexanoate (inter-I)

To a solution of (1S,2R,4S)-4-(2-amino-6-oxo-1H-purin-9(6H)-yl)-2-(((tert-butyldimethylsilyl)oxy)methyl)-3-methylenecyclopentyl hexanoate (2) (1.70 g, 3.47 mmol) in tetrahydrofuran (50 mL) was added 1 M TABF in tetrahydrofuran (10 mL, 10 mmol) at 0° C. The mixture was stirred at room temperature for 16 hours. The mixture was concentrated in vacuum to give a residue. The residue was purified by column C18 (Mobile Phase A: water, Mobile Phase B: acetonitrile, Gradient: 5-60% (% B)) to give the desired compound inter-I (470 mg, yield 36%) as white solids.

¹H NMR (400 MHZ, DMSO-d₆) δ 10.59 (s, 1H), 7.70 (s, 1H), 6.43 (br s, 2H), 5.35-5.26 (m, 2H), 5.19-5.12 (m, 1H), 5.03-4.94 (m, 1H), 4.60 (s, 1H), 3.66-3.56 (m, 2H), 2.71-2.63 (m, 1H), 2.56-2.42 (m, 1H), 2.34-2.29 (m, 2H), 2.25-2.14 (m, 1H), 1.61-1.47 (m, 2H), 1.36-1.19 (m, 4H), 0.97-0.92 (m, 3H).

### Example 20C: (1S,2R,4S)-4-(2-amino-6-oxo-1H-purin-9(6H)-yl)-3-methylene-2-((2-phenylacetoxy)methyl)cyclopentyl hexanoate (20)

To a mixture of (1S,2R,4S)-4-(2-amino-6-oxo-1H-purin-9(6H)-yl)-2-(hydroxymethyl)-3-methylenecyclopentyl hexanoate (inter-I) (200 mg, 0.530 mmol). N-(3-dimethylaminopropyl)-N′-ethylcarbodiimide hydrochloride (275 mg, 2.14 mmol), 4-dimethylaminopyridine (7 mg, 0.06 mmol) and 3-methylbutanoic acid (102 mg, 1.07 mmol) in N,N-dimethylformamide (20 mL) was added N,N-diisopropylethylamine (408 mg, 2.14 mmol) at 0° C. The mixture was stirred at room temperature for 16 hours. Then the mixture was poured into water (50 mL) and extracted with ethyl acetate (20 mL) for 3 times. The combined organic phases were washed with 0.5 M hydrochloride aqueous solution (20 mL), saturated sodium bicarbonate aqueous solution (20 mL) and brine (20 mL), and then dried over sodium sulfate and filtered. The filtrate was concentrated in vacuum. The residue was purified by column C18 (Mobile Phase A: water, Mobile Phase B: acetonitrile, Gradient: 5-60% (% B)) to give the desired 20 (125 mg, yield 51%) as white solid.

LC-MS (ESI): R_{T}=3.802 min, mass calcd. 459.2 for C₂₃H₃₃N₅O₅, m/z found 460.2 [M+H]⁺.

### Example 21

Compound 21 was prepared by using the procedure followed for the compound 20.

### Example 22: ((1R,2S,4S)-4-(2-amino-6-oxo-1H-purin-9(6H)-yl)-2-hydroxy-3-methylenecyclopentyl)methyl isobutyrate (22)

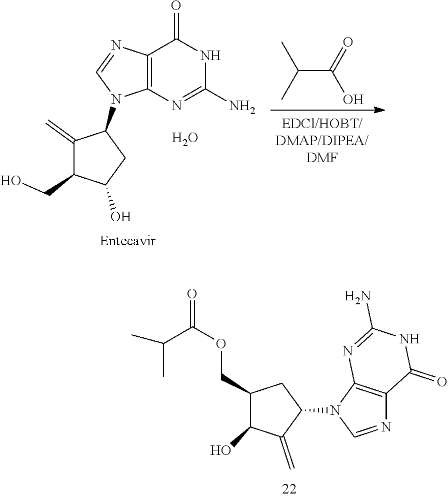

To a mixture of isobutyric acid (1.10 g, 12.4 mmol), 1-hydroxybenzotrizole (3.10 g, 23.2 mmol), 4-dimethylaminopyridine (0.2 g, 1.6 mmol) and N,N-diisopropylethylamine (6.00 g, 46.4 mmol) in N,N-dimethylformamide (30 mL) was added N-(3-dimethylaminopropyl)-N′-cthylcarbodiimide hydrochloride (4.40 g. 23.2 mmol) and Entecavir hydrate (4.60 g, 15.5 mmol) at 0° C. The mixture was stirred at room temperature for 16 hours. Then the mixture was poured into water (50 mL) and extracted with ethyl acetate (50 mL) for 3 times. The combined organic phases were washed with 0.5 M hydrochloride aqueous solution (5 mL), saturated sodium bicarbonate aqueous solution (50 mL) and brine (50 mL), and then dried over sodium sulfate and filtered. The filtrate was concentrated in vacuum. The residue was purified by column C18 (Mobile Phase A: water, Mobile Phase B: acetonitrile, Gradient: 5-80% (% B)) to give the desired 22 (1.7 g, yield 32%) as white solids.

LC-MS (ESI): R_{T}=2.255 min, mass calcd. 347.2 for C₁₆H₂₁N₅O₄, m/z found 348.1 [M+H]⁺.

### Examples 23-30

Compounds 23-30 were prepared by using the procedure followed for the compound 22 described above in Example 22.

### Example 31A: (1S,2R,4S)-4-(2-amino-6-oxo-1H-purin-9(6H)-yl)-2-(((tert-butyldimethylsilyl)oxy)methyl)-3-methylenecyclopentyl isobutyrate (inter-J)

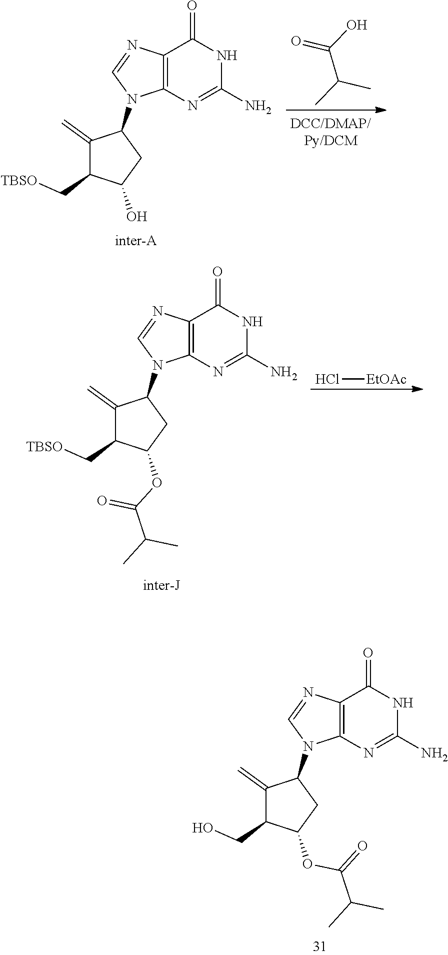

To a mixture of 2-amino-9-((1S,3R,4S)-3-(((tert-butyldimethylsilyl)oxy)methyl)-4-hydroxy-2-methylenecyclopentyl)-1H-purin-6(9H)-one (inter-A) (6.00 g, 15.3 mmol), isobutyric acid (2.03 g, 23.0 mmol), pyridine (14.6 g, 184 mmol) and 4-dimethylaminopyridine (187 mg, 1.53 mmol) in dichloromethane (300 mL) was added N,N′-dicyclohexylcarbodiimide (4.75 g, 23.0 mmol) at 0° C. After stirred at room temperature for 16 hours the mixture was filtered and the cake was washed with dichloromethane (50 mL). The filtrate was concentrated in vacuum. The residue was tritrurated in ethanol (250 mL). The mixture was filtered and the cake was washed with another 50 mL of dichloromethane to give a wet cake (10.5 g). The filtrate was concentrated in vacuum. The residue was purified by column C18 (Mobile Phase A: water, Mobile Phase B: acetonitrile, Gradient: 5-65% (% B)) to give the desired product. The wet cake and the new obtained product from pre-C18 were combined and dried in vacuum to give the desired inter-J (7.49 g, 85% purity, yield 90%) as white solids.

LC-MS (ESI): R_{T}=1.47 min, mass calcd. 461.3 for C₂₂H₃₅N₅O₄Si, m/z found 462.6 [M+H]⁺. ¹H NMR (400 MHz, DMSO-d₆) δ 10.51 (s, 1H), 7.57 (s, 1H), 6.36 (br s, 2H), 5.23 (t, J=8.4 Hz, 1H), 5.16-5.13 (m, 2H), 4.57 (s, 1H), 3.73-3.71 (m, 2H), 2.69-2.60 (m, 1H), 2.47-2.41 (m, 2H), 2.16-2.11 (m, 1H), 1.03-1.00 (m, 6H), 0.81 (s, 9H), 0.01 (s, 6H).

### Example 31B: (1S,2R,4S)-4-(2-amino-6-oxo-1H-purin-9(6H)-yl)-2-(hydroxymethyl)-3-methylenecyclopentyl isobutyrate (31)

To a mixture of (1S,2R,4S)-4-(2-amino-6-oxo-1H-purin-9(6H)-yl)-2-(((tert-butyldimethylsilyl)oxy)methyl)-3-methylenecyclopentyl isobutyrate (2) (7.45 g. 13.7 mmol) in ethyl acetate (30 mL) was added 4 M hydrochloride in ethyl acetate (30 mL) at room temperature. The mixture was stirred at room temperature for 2 hours. The mixture was concentrated in vacuum. The residue was triturated in ethanol (20 mL) to give the desired 31 (3.89 g, yield 82%) as white solids.

LC-MS (ESI): R_{T}=3.540 min, mass calcd. 347.2 for C₁₆H₂₁N₅O₄, m/z found 348.2 [M+H]⁺.

### Examples 32-37

Compounds 32-37 were prepared by using the procedure followed for the compound 32 described above in Example 31 with following variations: a) catalytic amount of p-toluenesulfonic acid (5 mg for 0.51 mmol reaction) used for 33 and 34; b) deprotection of TBS group was performing using i) TBAF for 33 ii) TFA for 34 iii) HCl-THF for 35 and 36.

### Example 38: Micronization of Selected Compounds

Selected compounds were micronized using Tecnologia Meccanica J20 with crushing pressure of 0.48 MPa and Inlet pressure of 0.38 MPa. All samples were crushed prior to jet milling. After micronization, the particle sizes are as follows: ETV (D₅₀=2.4 um: D₉₀=5.0 um); ETV-palmitate (D₅₀=3.0 um: D₉₀=10.5 um), prodrug 29 (D₅₀=1.8 um: D₉₀=4.0 um), and prodrug 36 (D₅₀=7.0 um: D₉₀=16.6 um).

### Pharmacokinetic (PK) Studies

Animals. Animals (Male SD rats ˜200-250 g and Male Beagle dog ˜12-15 kg) were obtained from an approved vendor.

Acclimation/Quarantine. Following arrival, animals were assessed as to their general health by a member of the veterinary staff or other authorized personnel. Animals were acclimated for at least 3 days before being placed on study.

Animal Husbandry. Animals were group housed during acclimation and individually housed during the study. The animal room environment was controlled (target conditions: temperature 18 to 26° ° C., relative humidity 30 to 70%, 12 hours artificial light and 12 hours dark). Temperature and relative humidity were monitored daily.

Animal Cannulation: No

Animals were non-fasted at least 12 hours prior to the administration. All animals had access to Certified Rodent and non-Rodent Diet ad libitum 4 hours post dosing.

Water was autoclaved before provided to the animals ad libitum. Periodic analyses of the water were performed, and the results archived. There are no known contaminants in the diet or water that, at the levels of detection, is expected to interfere with the purpose, conduct or outcome of the study.

Dose Formulation. IM/SC Formulation: The formulations were prepared according to the procedure given in Table 2-4. The formulations were prepared on the day of dosing. Animals were dosed within four hours after the formulation is prepared. Two 20 μL aliquots of each formulation were removed from each of the formulation solutions, transferred into 1.5 mL of polypropylene microcentrifuge tubes and run dose validation by LC/UV or LC-MS/MS.

Dose Administration. For SC/IM dosing, the dose formulation was administered via subcutaneous/intramuscular injection respectively following facility SOPs.

Sample Collection. Approximately 200 μL blood was collected from saphenous vein at each time point for rats and 0.5 mL for rhesus macaques. All blood samples were transferred into microcentrifuge tubes containing 4 μL of K₂EDTA (0.5M) as anti-coagulant and placed on wet ice until processed for plasma.

Blood/Plasma processing. Blood: Blood samples were processed for plasma by centrifugation at approximately 4° C., 3000 g 15 min within half an hour of collection. Plasma samples was stored in polypropylene tubes, quick frozen over dry ice and kept at −70±10° C. until LC/MS/MS analysis.

### Sample Analysis

### Dose Formulation Concentration Verification

Aliquots of the formulations were collected in the middle position of each dose formulation in duplicate
The concentrations of the test compound in dose formulation samples were determined by the LC/UV or LC/MS/MS method

### Bioanalytical Method and Sample Analysis

LC-MS/MS methods for the quantitative determination of test compound in corresponded biological matrix was developed under non-GLP compliance.
A calibration curve with 8 non-zero calibration standards was applied for the method including LLOQ.
A set of QC samples consisting of low, middle, and high concentration was applied for the method.
The study sample analysis will be performed concurrently with a set of calibration standards and two sets of QC samples using the LC-MS/MS method (If sample numbers were more than 48, then two calibration curves with 2 sets of QC samples were applied).
Acceptance criteria:
   Linearity: a minimum of 6 calibration standards was back calculated to within ±20% of their nominal values in plasma.
   Accuracy: A minimum of 4 out of 6 QC samples was back calculated to within ±20% of their nominal values in plasma.
   Specificity: The mean calculated concentration in the single blank matrix should be 0.5 times the LLOQ.
   Sensitivity: the LLOQ will be tried to target 1˜3 ng/mL.
   Carryover: the mean calculated carry-over concentration in the single blank matrix immediately after the highest standard injection should be LLOQ. If the carryover couldn't meet the criteria, then the percent of carryover should be estimated following in-house bioanalytical SOP.

Data Analysis. Plasma concentration versus time data was analyzed by non-compartmental approaches using the Phoenix WinNonlin 6.3 software program. Cₘₐₓ, Tₘₐₓ, T_{1/2}, AUC₍₀₋₁₎, AUC₍₀₋ᵢₙₜ₎, MRT₍₀₋₁₎, MRT₍₀₋ᵢₙₜ₎, % F and graphs of plasma concentration versus time profile were reported.

### Example 39

Bis-DHA prodrug 9 (31 mg/kg equivalent dose of ETV) and ETV (20 mg/kg) were subjected to single dose rat PK studies via intramuscular route of administration. Non-micronized prodrug and ETV were used. Prodrug 9 provided a delayed and 248-fold lower Cₘₐₓ than ETV. Enhanced half-life and mean residence life were also observed for prodrug 9 making it suitable as a long-acting agent.

Table 2 shows the rat PK data for ETV and prodrug 9 following IM administration. The data are shown in graphic form in FIG. 18.

TABLE 2

| | IM | IM |
|---|---|---|
| | ETV | Prodrug 9 |
| Dose of ETV (mg/kg) | 20 | 31 |
| Formulation | Sesame oil suspension | Castor oil solution |
| Conc of ETV (mg/mL) | 100 | 77 |
| T_{1/2} (h) | 20.8 ± 7.9 | 330 ± 36 |
| MRT₀₋ₗₐₛₜ (h) | 6.6 ± 1.3 | 237 ± 5 |
| Tₘₐₓ (h) | 1 ± 0 | 24 ± 0 |
| Cₘₐₓ (ng/mL) | 1833 ± 323 | 11.6 ± 1.4 |
| d.n. Cₘₐₓ (ng/mL/mg/kg) | 91.65 | 0.37 |
| Cₗₐₛₜ (ng/mL) | 1.4 (5 day) | 1.9 (28 day) |
| AUC₀₋ₗₐₛₜ (ng/mL*h) | 7107 ± 1558 | 2829 ± 85 |
| AUC_{0-inf} (ng/mL*h) | 7149 ± 1582 | 3726 ± 318 |

### Example 40

Prodrugs 11 and 29, and ETV, were subjected to single dose dog PK studies via intramuscular injection with 20 mg/kg equivalent dose of ETV and were monitored for 112 days (see table 3). The aqueous suspension formulations were derived from 0.5% CMC-Na and 0.5% Tween 80. Prodrug 6 exhibited plasma levels of ETV above 0.5 ng/ml for almost >112 days. On the contrary, Entecavir was found to be below 0.5 ng/ml@day 23. The monoester-based prodrug 29 provided therapeutic levels up to 55 days. Micronized ETV (D₅₀=2.4 um; D₉₀=5.0 um) and prodrug 29 (D₅₀=1.8 um; D₉₀=4.0 um) and non-micronized prodrug 11 (D₅₀=4.4 um; D₉₀=13.0 um) were used for this study.

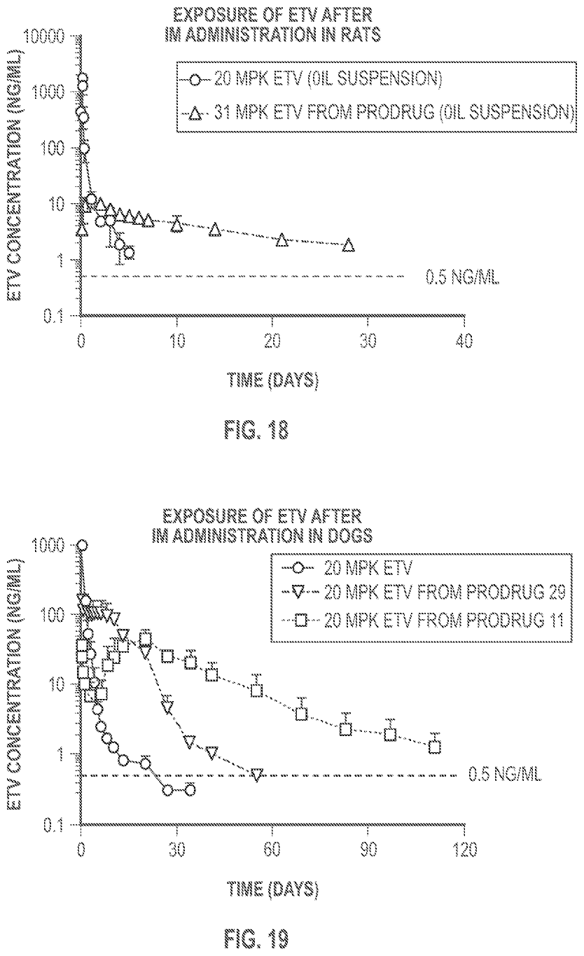

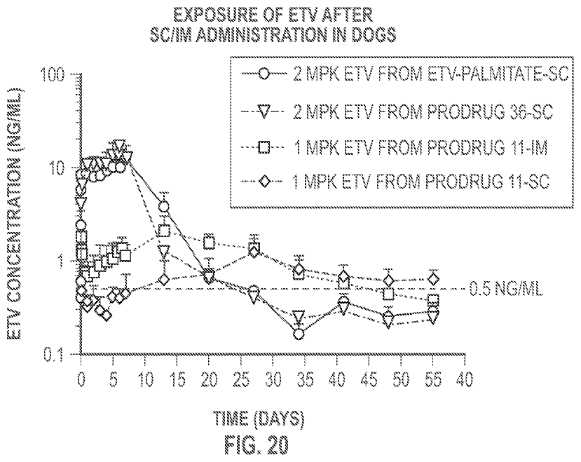

Table 3 shows the Dog PK data for prodrug 11, 29 and ETV following IM administration. The data are shown in graphic form in FIG. 19.

TABLE 3

| | IM | IM | IM |
|---|---|---|---|
| | ETV | Prodrug 29 | Prodrug 11 |
| Dose of ETV (mg/kg) | 20 | 20 | 20 |
| Formulation | 0.5% CMC-Na/0.5% Tween 80 | | |
| Conc of ETV (mg/mL) | 100 | 71 | 54 |
| T_{1/2} (h) | 438 ± 263 | 327 ± 44 | 648 ± 201 |
| MRT_{0-inf} (h) | 30.8 ± 4.8 | 234 ± 64 | 817 ± 150 |
| Tₘₐₓ (h) | 2.3 ± 1.2 | 18.3 ± 9.8 | 480 ± 0.0 |
| Cₘₐₓ (ng/mL) | 2390 ± 219 | 145 ± 58.5 | 44.8 ± 10.0 |
| Cₗₐₛₜ (ng/mL) | 0.2 (41 day) | 0.5 (55 day) | 1.3 (111 day) |
| AUC₀₋ₗₐₛₜ (ng/mL*h) | 25134 ± 1970 | 39503 ± 3537 | 32178 ± 6823 |
| AUC_{0-inf} (ng/mL*h) | 25294 ± 2044 | 39732 ± 3486 | 33232 ± 7243 |

### Example 41

Prodrugs 11 and 36, and ETV-palmitate, were subjected to a single dose dog PK studies via intramuscular and subcutaneous route of administration with various equivalent doses of ETV (˜1-2 mg/kg) and were monitored for 56 days (see table 4). The aqueous suspension formulations were derived from 0.5% CMC-Na and 0.5% Tween 80. Prodrug 6 exhibited plasma levels of ETV above 0.5 ng/mL for almost 45 days@1 mg/kg ETV equivalent dose when administered via IM route. On the contrary, known prodrug, ETV-palmitate provided plasma levels above 0.5 ng/ml for ˜27 days only with double equivalent dose of ETV. SC route administration of prodrug 6 provides concentrations above 0.5 ng/mL for more than 56 days along with initial slow absorption. Micronized ETV-palmitate (D₅₀=3.0 um; D₉₀=10.5 um) and prodrug 36 (D₅₀=7.0 um; D₉₀=16.6 um) and non-micronized prodrug 11 (D₅₀=4.4 um; D₉₀=13.0 um) were used for this study.

Table 4 shows the Dog PK data for prodrug 11, 36 and ETV-palmitate following IM/SC administration. The data are shown in graphic form in FIG. 12.

TABLE 4

| | SC | SC | SC | IM |
|---|---|---|---|---|
| | ETV-palmitate | Prodrug 36 | Prodrug 11 | Prodrug 11 |
| Dose of ETV (mg/kg) | 2.15 | 2.1 | 1.08 | 1.08 |
| Formulation | 0.5% CMC-Na/0.5% Tween 80 | | | |
| Conc of ETV | 54 | 70 | 54 | 54 |
| (mg/mL) | | | | |
| T_{1/2} (h) | 352 ± 64 | 639 ± 53 | 1047 ± 160 | 392 ± 94 |
| MRT_{0-inf} (h) | 293 ± 37 | 318 ± 27 | 1780 ± 244 | 708 ± 74 |
| Tₘₐₓ (h) | 152 ± 28 | 120 ± 42 | 216 ± 374 | 208 ± 180 |
| Cₘₐₓ (ng/mL) | 13.5 ± 3.3 | 17.3 ± 7.0 | 1.9 ± 0.5 | 2.4 ± 0.5 |
| d.n. Cₘₐₓ (ng/mL/ | 6.3 | 8.2 | 1.8 | 2.2 |
| mg/kg) | | | | |
| Cₗₐₛₜ (ng/mL) | 0.2 (41 day) | 0.2 (55 day) | 0.6 (55 day) | 0.4 (55 day) |
| AUC₀₋ₗₐₛₜ (ng/mL*h) | 3342 ± 404 | 3330 ± 692 | 944 ± 191 | 1389 ± 115 |
| d.n. AUC₀₋ₗₐₛₜ | 1554 | 1586 | 874 | 1286 |
| (ng/mL*h/mg/kg) | | | | |
| AUC_{0-inf} (ng/mL*h) | 3489 ± 400 | 3947 ± 697 | 1931 ± 506 | 1661 ± 182 |

### Example 42

For powder X-ray analyses, an appropriate amount of sample compound powder (generally about 20 to 50 mg) was placed on a sample pan, and gently compressed with a glass plate to yield a flat surface for X-ray scanning. Samples generally were not ground and sieved to avoid resultant external forces from causing any crystal transition. For some block samples, a small scraper was used to press into powder. Data collection was performed on a Shimadzu XRD-6000 diffractometer using CuK-alpha radiation (λ=1.54056 Å); voltage at 40 kV and current at 30 mA; and scan scope from 2 to 60 degrees at 5 deg./min.

We claim:

1. A compound of formula (I) or a pharmaceutically acceptable salt thereof: 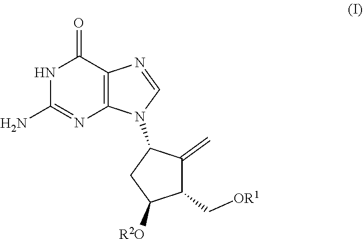
wherein
R¹ is H or is X-Lₘ,
m=1 or 2;
X-Lₘ is —C(═O)L, —C(═O)OL, —C(═O)NH(L), —C(═O)N(L)₂, —CH(R)OC(═O)L, —C(═O)CH(R)—NH(L), —C(═O)CH(R)—N(L)₂, —P(═O)(NHL)₂, —P(═O)(NHL)(NL₂), or —P(═O)(NL₂)₂,
each L is independently selected from (C1-22, linear and branched)alkyl, (C3-22, linear and branched)alkenyl, (C3-7)cycloalkyl, (CHR)ₙ-phenyl wherein n=0 or 1, and —CHR—N(R)₂;
or R¹ is —OCH(R)OP(═O)(OH)₂, a phosphate residue or its derivative residue comprising a monophosphate, a diphosphate, a triphosphate, a phosphonate, a phosphate polyester, a phosphate monoamidate, a phosphate diamidate, a phosphorothioate, a phosphoroselenoate, or a phosphoroboranoate;
R is H, (C1-22)alkyl, (C3-22)alkenyl, (C3-7)cycloalkyl, or (C6-C12)aryl;
R² is H or is X-Lₘ;
or R² is —OCH(R)OP(═O)(OH)₂, a phosphate residue or its derivative residue comprising a monophosphate, a diphosphate, a triphosphate, a phosphonate, a phosphate polyester, a phosphate monoamidate, a phosphate diamidate, a phosphorothioate, a phosphoroselenoate, or a phosphoroboranoate,
and wherein R¹ and R² are not simultaneously H.

2. The compound or pharmaceutically acceptable salt thereof according to claim 1, wherein each of R¹ and R² is X-Lₘ.

3. The compound or pharmaceutically acceptable salt thereof according to claim 1 or 2. wherein X-Lₘ is —C(═O)L.

4. A compound or a pharmaceutically acceptable salt thereof, wherein the compound is selected from the following table:

| | |
|---|---|
| 1 | 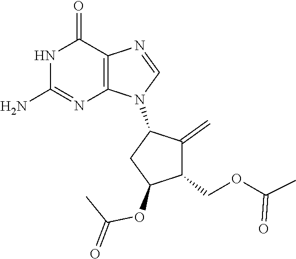 [SMILES: C=C1[C@H](COC(C)=O)[C@@H](OC(C)=O)C[C@@H]1n1cnc2c(=O)[nH]c(N)nc21] |
| 2 | 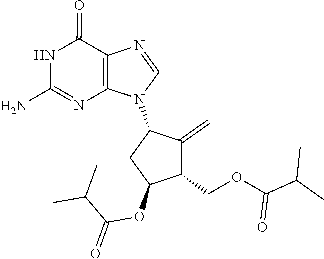 [SMILES: C=C1[C@H](COC(=O)C(C)C)[C@@H](OC(=O)C(C)C)C[C@@H]1n1cnc2c(=O)[nH]c(N)nc21] |
| 3 | 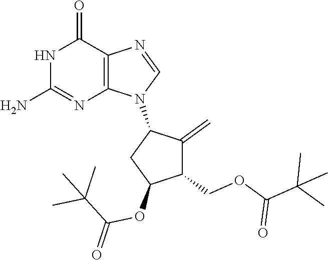 [SMILES: C=C1[C@H](COC(=O)C(C)(C)C)[C@@H](OC(=O)C(C)(C)C)C[C@@H]1n1cnc2c(=O)[nH]c(N)nc21] |
| 4 | 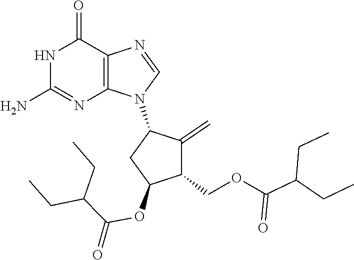 [SMILES: C=C1[C@H](COC(=O)C(CC)CC)[C@@H](OC(=O)C(CC)CC)C[C@@H]1n1cnc2c(=O)[nH]c(N)nc21] |
| 5 | 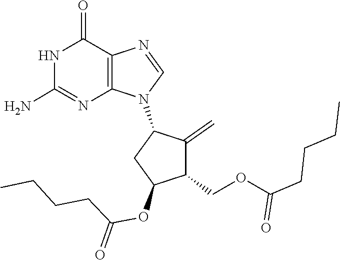 [SMILES: C=C1[C@H](COC(=O)CCCC)[C@@H](OC(=O)CCCC)C[C@@H]1n1cnc2c(=O)[nH]c(N)nc21] |
| 6 | 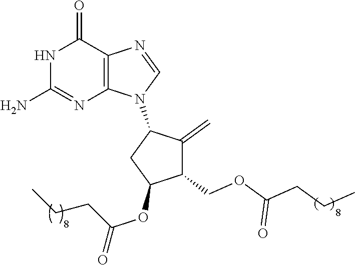 |
| 7 | 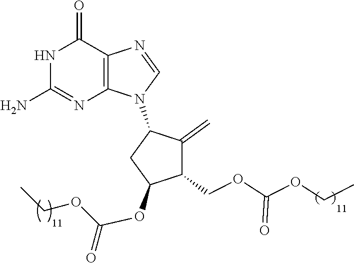 |
| 8 | 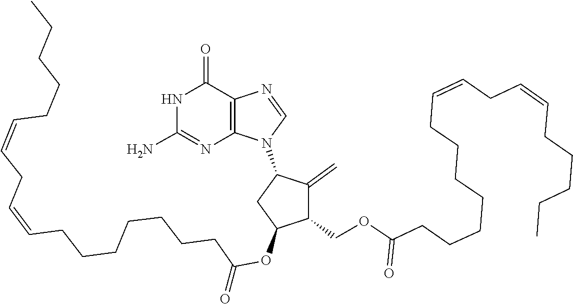 [SMILES: C=C1[C@H](COC(=O)CCCCCCC/C=C\C/C=C\CCCCC)[C@@H](OC(=O)CCCCCCC/C=C\C/C=C\CCCCC)C[C@@H]1n1cnc2c(=O)[nH]c(N)nc21] |
| 9 | 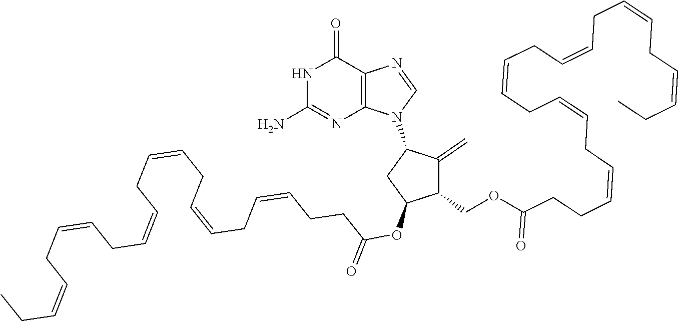 [SMILES: C=C1[C@H](COC(=O)CC/C=C\C/C=C\C/C=C\C/C=C\C/C=C\C/C=C\CC)[C@@H](OC(=O)CC/C=C\C/C=C\C/C=C\C/C=C\C/C=C\C/C=C\CC)C[C@@H]1n1cnc2c(=O)[nH]c(N)nc21] |
| 10 | 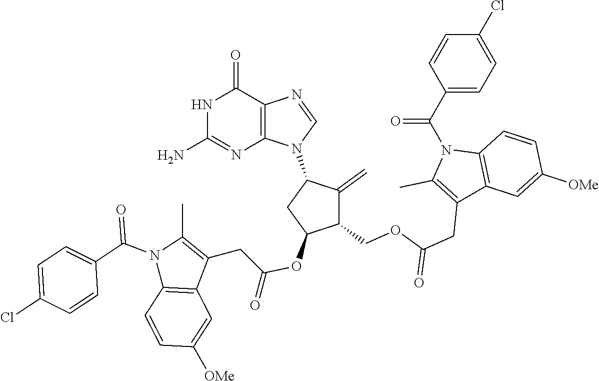 [SMILES: C=C1[C@H](COC(=O)Cc2c(C)n(C(=O)c3ccc(Cl)cc3)c3ccc(OC)cc23)[C@@H](OC(=O)Cc2c(C)n(C(=O)c3ccc(Cl)cc3)c3ccc(OC)cc23)C[C@@H]1n1cnc2c(=O)[nH]c(N)nc21] |
| 11 | 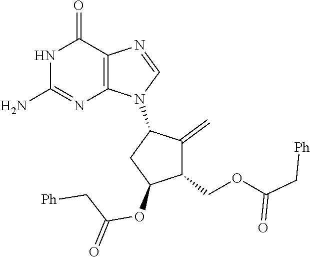 [SMILES: C=C1[C@H](COC(=O)Cc2ccccc2)[C@@H](OC(=O)Cc2ccccc2)C[C@@H]1n1cnc2c(=O)[nH]c(N)nc21] |
| 12 | 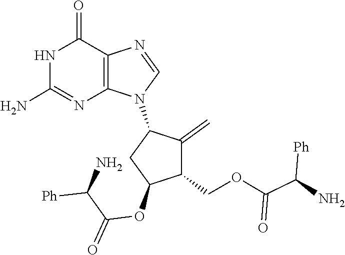 [SMILES: C=C1[C@H](COC(=O)[C@H](N)c2ccccc2)[C@@H](OC(=O)[C@H](N)c2ccccc2)C[C@@H]1n1cnc2c(=O)[nH]c(N)nc21] |
| 13 | 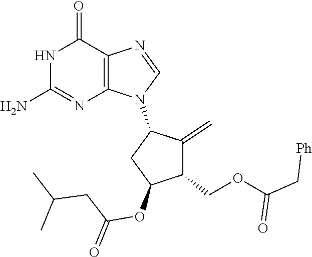 [SMILES: C=C1[C@H](COC(=O)Cc2ccccc2)[C@@H](OC(=O)CC(C)C)C[C@@H]1n1cnc2c(=O)[nH]c(N)nc21] |
| 14 | 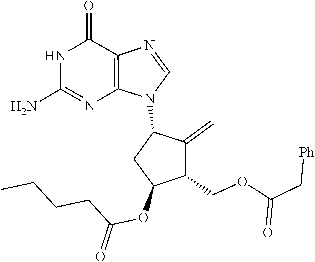 [SMILES: C=C1[C@H](COC(=O)Cc2ccccc2)[C@@H](OC(=O)CCCC)C[C@@H]1n1cnc2c(=O)[nH]c(N)nc21] |
| 15 | 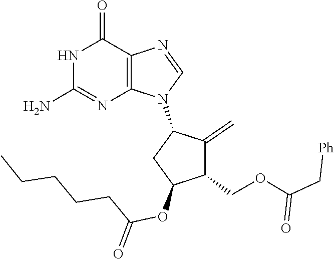 [SMILES: C=C1[C@H](COC(=O)Cc2ccccc2)[C@@H](OC(=O)CCCCC)C[C@@H]1n1cnc2c(=O)[nH]c(N)nc21] |
| 16 | 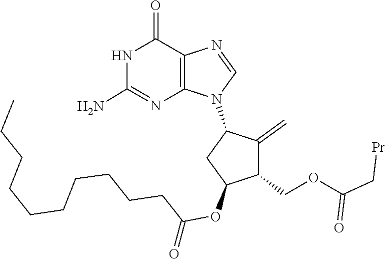 |
| 17 | 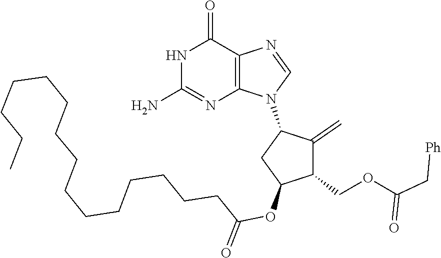 [SMILES: C=C1[C@H](COC(=O)Cc2ccccc2)[C@@H](OC(=O)CCCCCCCCCCCCCCC)C[C@@H]1n1cnc2c(=O)[nH]c(N)nc21] |
| 18 | 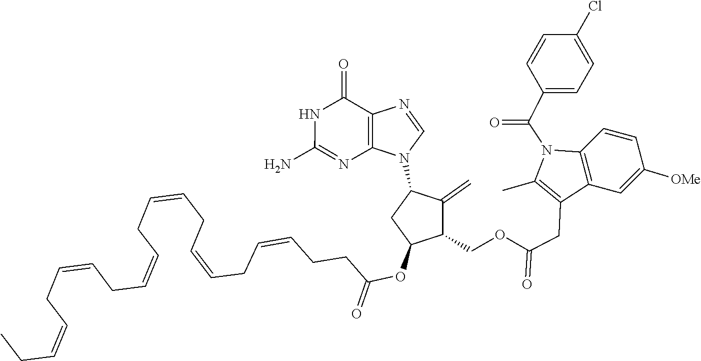 [SMILES: C=C1[C@H](COC(=O)Cc2c(C)n(C(=O)c3ccc(Cl)cc3)c3ccc(OC)cc23)[C@@H](OC(=O)CC/C=C\C/C=C\C/C=C\C/C=C\C/C=C\C/C=C\CC)C[C@@H]1n1cnc2c(=O)[nH]c(N)nc21] |
| 19 | 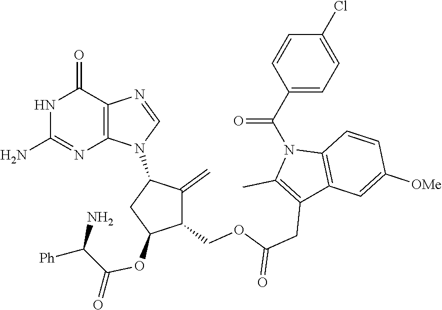 [SMILES: C=C1[C@H](COC(=O)Cc2c(C)n(C(=O)c3ccc(Cl)cc3)c3ccc(OC)cc23)[C@@H](OC(=O)[C@H](N)c2ccccc2)C[C@@H]1n1cnc2c(=O)[nH]c(N)nc21] |
| 20 | 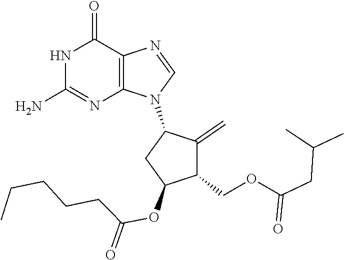 [SMILES: C=C1[C@H](COC(=O)CC(C)C)[C@@H](OC(=O)CCCCC)C[C@@H]1n1cnc2c(=O)[nH]c(N)nc21] |
| 21 | 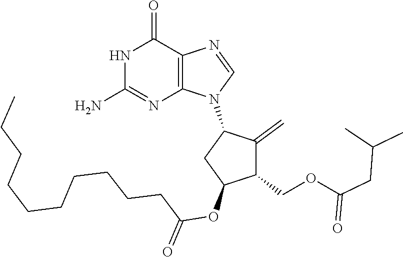 [SMILES: C=C1[C@H](COC(=O)CC(C)C)[C@@H](OC(=O)CCCCCCCCCC)C[C@@H]1n1cnc2c(=O)[nH]c(N)nc21] |
| 22 | 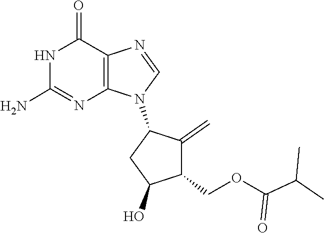 [SMILES: C=C1[C@H](COC(=O)C(C)C)[C@@H](O)C[C@@H]1n1cnc2c(=O)[nH]c(N)nc21] |
| 23 | 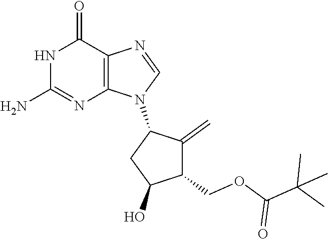 [SMILES: C=C1[C@H](COC(=O)C(C)(C)C)[C@@H](O)C[C@@H]1n1cnc2c(=O)[nH]c(N)nc21] |
| 24 | 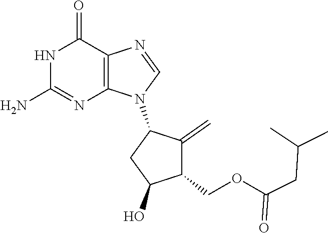 [SMILES: C=C1[C@H](COC(=O)CC(C)C)[C@@H](O)C[C@@H]1n1cnc2c(=O)[nH]c(N)nc21] |
| 25 | 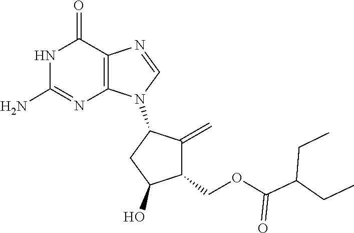 [SMILES: C=C1[C@H](COC(=O)C(CC)CC)[C@@H](O)C[C@@H]1n1cnc2c(=O)[nH]c(N)nc21] |
| 26 | 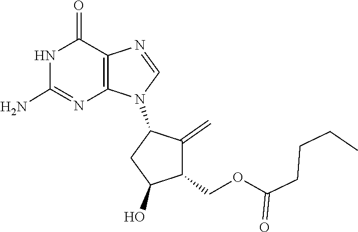 [SMILES: C=C1[C@H](COC(=O)CCCC)[C@@H](O)C[C@@H]1n1cnc2c(=O)[nH]c(N)nc21] |
| 27 | 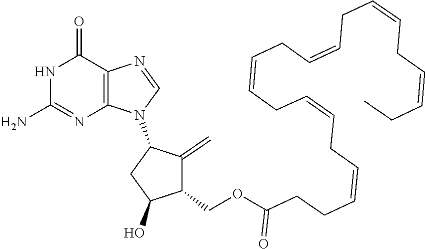 [SMILES: C=C1[C@H](COC(=O)CC/C=C\C/C=C\C/C=C\C/C=C\C/C=C\C/C=C\CC)[C@@H](O)C[C@@H]1n1cnc2c(=O)[nH]c(N)nc21] |
| 28 | 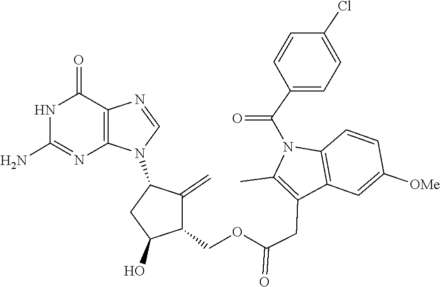 [SMILES: C=C1[C@H](COC(=O)Cc2c(C)n(C(=O)c3ccc(Cl)cc3)c3ccc(OC)cc23)[C@@H](O)C[C@@H]1n1cnc2c(=O)[nH]c(N)nc21] |
| 29 | 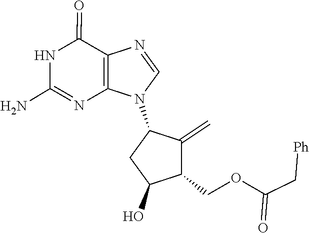 [SMILES: C=C1[C@H](COC(=O)Cc2ccccc2)[C@@H](O)C[C@@H]1n1cnc2c(=O)[nH]c(N)nc21] |
| 30 | 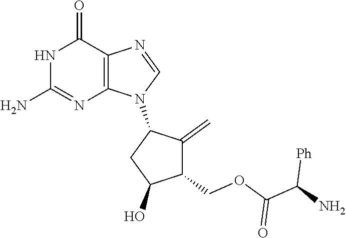 [SMILES: C=C1[C@H](COC(=O)[C@H](N)c2ccccc2)[C@@H](O)C[C@@H]1n1cnc2c(=O)[nH]c(N)nc21] |
| 31 | 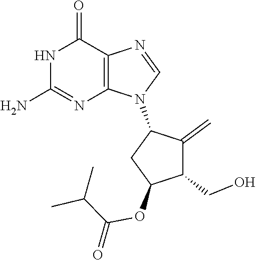 [SMILES: C=C1[C@H](CO)[C@@H](OC(=O)C(C)C)C[C@@H]1n1cnc2c(=O)[nH]c(N)nc21] |
| 32 | 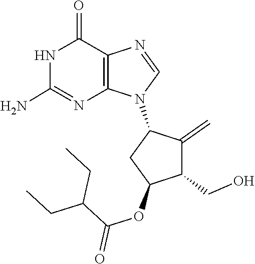 [SMILES: C=C1[C@H](CO)[C@@H](OC(=O)C(CC)CC)C[C@@H]1n1cnc2c(=O)[nH]c(N)nc21] |
| 33 | 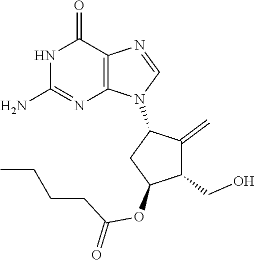 [SMILES: C=C1[C@H](CO)[C@@H](OC(=O)CCCC)C[C@@H]1n1cnc2c(=O)[nH]c(N)nc21] |
| 34 | 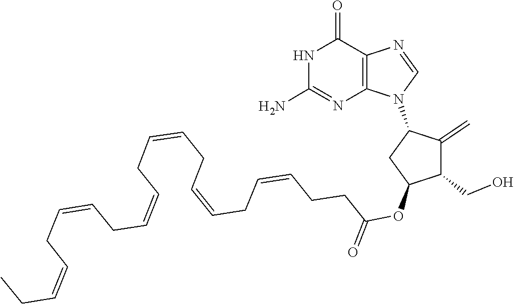 [SMILES: C=C1[C@H](CO)[C@@H](OC(=O)CC/C=C\C/C=C\C/C=C\C/C=C\C/C=C\C/C=C\CC)C[C@@H]1n1cnc2c(=O)[nH]c(N)nc21] |
| 35 | 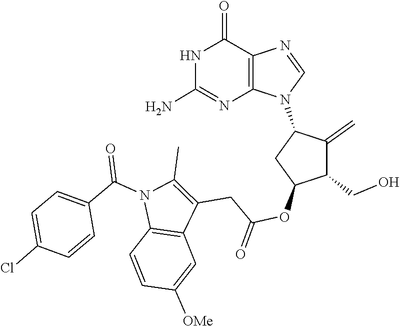 [SMILES: C=C1[C@H](CO)[C@@H](OC(=O)Cc2c(C)n(C(=O)c3ccc(Cl)cc3)c3ccc(OC)cc23)C[C@@H]1n1cnc2c(=O)[nH]c(N)nc21] |
| 36 | 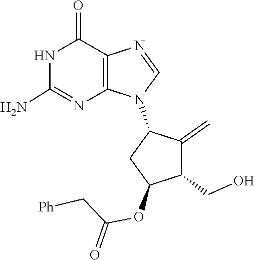 [SMILES: C=C1[C@H](CO)[C@@H](OC(=O)Cc2ccccc2)C[C@@H]1n1cnc2c(=O)[nH]c(N)nc21] |
| 37 | 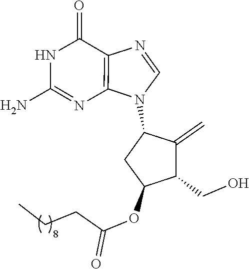 |
| 38 | 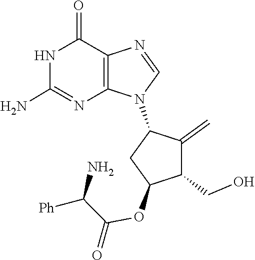 [SMILES: C=C1[C@H](CO)[C@@H](OC(=O)[C@H](N)c2ccccc2)C[C@@H]1n1cnc2c(=O)[nH]c(N)nc21] |

5. Compound 11 according to claim 4: 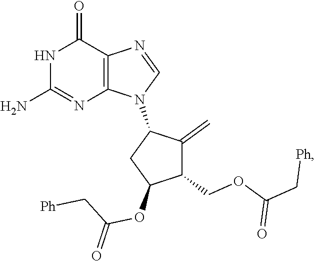 [SMILES: C=C1[C@H](COC(=O)Cc2ccccc2)[C@@H](OC(=O)Cc2ccccc2)C[C@@H]1n1cnc2c(=O)[nH]c(N)nc21]
wherein the compound is in crystalline form and is characterized by an X-ray powder diffractogram comprising the following peaks: 9.00, 17.28, 21.72, and 23.80°2θ±0.20°2θ as determined on a diffractometer using Cu—Kα₁ radiation at a wavelength of 1.54056 Å.

6. The compound according to claim 5, wherein the diffractogram further comprises the following peaks: 11.74, 19.48, 25.00, and 27.16°2θ+0.20°2θ.

7. Compound 36 according to claim 4: 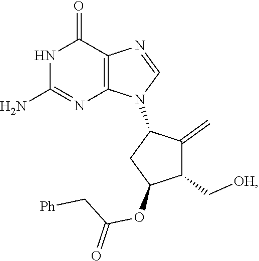 [SMILES: C=C1[C@H](CO)[C@@H](OC(=O)Cc2ccccc2)C[C@@H]1n1cnc2c(=O)[nH]c(N)nc21]
wherein the compound is in crystalline form and is characterized by an X-ray powder diffractogram comprising the following peaks: 16.44, 19.36, 20.88, and 26.46°2θ±0.20°2θ as determined on a diffractometer using Cu—Kα₁ radiation at a wavelength of 1.54056 Å.

8. The compound according to claim 7, wherein the diffractogram further comprises the following peaks: 15.18, 22.16, 24.56, and 28.58°2θ±0.20°2θ.

9. Compound 29 according to claim 4: 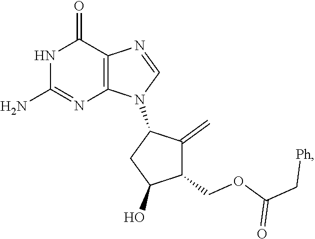 [SMILES: C=C1[C@H](COC(=O)Cc2ccccc2)[C@@H](O)C[C@@H]1n1cnc2c(=O)[nH]c(N)nc21]
wherein the compound is in crystalline form and is characterized by an X-ray powder diffractogram comprising the following peaks: 12.30, 18.62, 20.34, and 25.54°2θ+0.20°2θ as determined on a diffractometer using Cu—Kα₁ radiation at a wavelength of 1.54056 Å.

10. The compound according to claim 9, wherein the diffractogram further comprises the following peaks: 14.96, 16.54, 21.38, and 27.74°2θ+0.20°2θ.

11. A pharmaceutical composition comprising an effective amount of a compound or a pharmaceutically acceptable salt thereof according to any one of claims 1-10, and a pharmaceutically acceptable carrier.

12. The pharmaceutical composition according to claim 11, wherein the pharmaceutically acceptable carrier comprises sesame oil, castor oil, or combinations thereof.

13. A formulation comprising a compound or pharmaceutically acceptable salt thereof according to any one of claims 1-10, wherein the compound is suspended in an aqueous solution comprising a water-soluble cellulose-based polymer and a non-ionic surfactant.

14. The formulation according to claim 13, wherein the water-soluble cellulose-based polymer is selected from the group consisting of alkyl celluloses, hydroxyalkyl celluloses. alkyl hydroxyalkyl celluloses, carboxyalkyl celluloses and salts thereof, and combinations thereof.

15. The formulation according to claim 13 or 14, wherein the water-soluble cellulose-based polymer is selected from the group consisting of methyl cellulose, ethyl cellulose, propyl cellulose, hydroxymethyl cellulose, hydroxyethyl cellulose, hydroxypropyl cellulose, bydroxypropylmethyl cellulose, carboxymethyl cellulose and salts thereof, and combinations thereof.

16. The formulation according to any one of claims 13-15, wherein the water-soluble cellulose-based polymer is sodium carboxymethyl cellulose (CMC).

17. The formulation according any one of claims 13 to 16, wherein the non-ionic surfactant is selected from the group consisting of polyoxyethylene fatty acid esters, sorbitan fatty acid ester anhydrides, polyoxyethylene sorbitan esters, polyoxyethylene alkyl ethers, polyoxyethylene alkyl phenol ethers, polyoxyethylene fatty amines, polyoxyethylene alkyl amides, sorbitol anhydride fatty acid esters, and polyoxyethylene ethers.

18. The formulation according any one of claims 13-17, wherein the non-ionic surfactant is selected from polyoxyethylene sorbitan esters.

19. The formulation according to any one of claims 13-18, wherein the water-soluble cellulose-based polymer is present in an amount of about 0.1 to about 2.0% (w/v).

20. The formulation according to any one of claims 13-19, wherein the water-soluble cellulose-based polymer is present in amount of about 0.3 to about 0.8% (w/v).

21. The formulation according to any one of claims 13-20, wherein the water-soluble cellulose-based polymer is present in amount of about 0.5% (w/v).

22. The formulation according to any one of claims 13-21, wherein the non-ionic surfactant is present in amount of about 0.1 to about 2.0% (w/v).

23. The formulation according to any one of claims 13-22, wherein the non-ionic surfactant is present in amount of about 0.3 to about 0.8% (w/v).

24. The formulation according to any one of claims 13-23, wherein the non-ionic surfactant is present in amount of about 0.5% (w/v).

25. The formulation according to any one of claims 13-24, wherein the water-soluble cellulose-based polymer is CMC in an amount of about 0.5% (w/v) and the non-ionic surfactant is Tween-80 in an amount of about 0.5% (w/v).

26. A method of inhibiting viral reverse transcriptase bioactivity, comprising contacting a virus expressing an enzyme with reverse transcriptase bioactivity with an effective amount or concentration of a compound or a pharmaceutically acceptable salt thereof according to any one of claims 1-10.

27. A method of treatment of a viral infection in a patient wherein inhibition of a reverse transcriptase is medically indicated, comprising administering to the patient an effective amount or concentration of a compound or a pharmaceutically acceptable salt thereof according to any one of claims 1-10.

28. A method for treatment or prevention of a viral infection in a patient, comprising administering to the patient in need of treatment or prevention an effective amount of a compound or a pharmaceutically acceptable salt thereof according to any one of claims 1-10.

29. The method according to claim 27 or 28, wherein the administering is selected from oral, parenteral, and implant routes of administration.

30. The method according to any one of claims 27-29, wherein the compound is formulated as an aqueous suspension or a solution, or is encapsulated in particles comprising poly(lactic-co-glycolic acid) (PLGA) for slow-release.

31. The method of any one of claims 27-30, wherein the viral infection is caused by hepatitis B virus (HBV).

32. The method of any one of claims 27-31, further comprising administering at least one anti-HBV agent.

33. The method of claim 32, wherein the anti-HBV agent is selected from adefovir dipivoxil, telbivudine, tenofovir disoproxil fumarate, tenofovir alafenamide fumarate, lamivudine, and combinations thereof.

34. The compound or pharmaceutically acceptable salt thereof according to any one of claims 1-10 for the treatment or prevention of a viral infection of HBV.
